SEQUENCE LISTING (21 sequences)

SEQ ID NO: 1 - protein, 143 aa, Oryctolagus cuniculus
  MISC_FEATURE: 5H2 heavy chain variable region
METGLRWLLLVAVLKGVQCQSVKESEGGLFKPTDTLTLTCTVSGFSLYSGAINWVRQAPGEGLQYIGWISDVGSAYYASWAKSRSTITRNTNENTVTLKMTSLTAADTATYFCARGDGSSGNYWVTDIWGQGTLVTVSSGQPK

SEQ ID NO: 2 - protein, 132 aa, Oryctolagus cuniculus
  MISC_FEATURE: 5K3 light chain variable region
MDTRAPTQLLGLLLLWLPGARCAVVLTQTASPVSAPVGGTVTINCQASESISSRLAWYQQKPGQPPKLLIYGASTLASGVPSRFKGSGSGTEFTLTISDLECADAATYSCQEYLSDNTFGGGTEVVVKGDPV

SEQ ID NO: 3 - protein, 143 aa, Oryctolagus cuniculus; identical to SEQ ID NO: 1
  MISC_FEATURE: 8H3 heavy chain variable region

SEQ ID NO: 4 - protein, 132 aa, Oryctolagus cuniculus; identical to SEQ ID NO: 2
  MISC_FEATURE: 8K3 light chain variable region

SEQ ID NO: 5 - protein, 138 aa, Oryctolagus cuniculus
  MISC_FEATURE: 9H3 heavy chain variable region
METGLRWLLLVAVLKGVQCQSVKESEGGLFKPTDTLTLTCTVSGFSLNSYGVNWVRQAPGNGLEYIGTVGSSGSAYYASWAKSRSTITRNTNLNTVTLKMTSLTAADTATYFCARGLIATMSIWGQGTLVTVSSGQPK

SEQ ID NO: 6 - protein, 136 aa, Oryctolagus cuniculus
  MISC_FEATURE: 9K2 light chain variable region
MDTRAPTQLLGLLLLWLPGATFAQVLTQTASSVSAAVGGTVTISCQSSQSVHSNNYLAWYQQKPGQPPKQLIYLASTLASGVPSRFKGSGSGTQFTLTISDLECDDAATYYCAGGYSGMIFTFGGGTEVVVKGDPV

SEQ ID NO: 7 - protein, 144 aa, Oryctolagus cuniculus
  MISC_FEATURE: 14H1 heavy chain variable region
METGLRWLLLVAVLKGVQCQSLEESGGDLVKPGAFLTLTCTASGFSFSGSDYMWWVRQAPGKGLEWIACIYGGYSGSTYYASWAKGRFTISKTSSTTVTLQMTRLTAADTAIYFCARDGGVTHFSHFDLWGQGTLVTVSSGQPK

SEQ ID NO: 8 - protein, 136 aa, Oryctolagus cuniculus
  MISC_FEATURE: 14K1 light chain variable region
MDTRAPTQLLGLLLLWLPGARCDVVMTQTPASVSEPVGGTVTIKCQASEDIYRLLAWYQQKPGQPPKLLIYGASTLASGVPSRFSGSGSGTEYTLTINDLECADAATYYCQYITYGSDVLTAFGGGTEVVVKGDPV

SEQ ID NO: 9 - protein, 145 aa, Oryctolagus cuniculus
  MISC_FEATURE: 15H1 heavy chain variable region
METGLRWLLLVAVLKGVQCQSLEESGGDLVKPGASLTLTCTASGFSFSRSYYVCWVRQAPGKGLEWIVCIYGGSSDTTYYASWAKGRFTISKTSSTTVTLQLNSLTAADTATYFCARRDVSGGYDYGMDLWGQGTLVTVSSGQPK

SEQ ID NO: 10 - protein, 136 aa, Oryctolagus cuniculus
  MISC_FEATURE: 15K3 light chain variable region
MDTRAPTQLLGLLLLWLPGARCAYDMTQTPASVEVAVGGTVTIKCQASEDIESYLAWYQQKPGQPPKLLIYDASDLASGVPSRFKGSGSGTEYTLTISDLECDDAATYYCRQGYSSSNVDNVFGGGTEVVVKGDPV

SEQ ID NO: 11 - protein, 144 aa, Oryctolagus cuniculus
  MISC_FEATURE: 24H1 heavy chain variable region
METGLRWLLLVAVLKGVQCQSLEESGGDLVKPGASLTLTCIGSGFDFSSNAIWWVRQAPGKGLEWIACLYGGTSGSTEYATWAKGRFTISKTSSTTVTLQMTSLTDADTATYYCAGGVVTWSYPRQLYLWGQGTLVTVSSGQPK

SEQ ID NO: 12 - protein, 135 aa, Oryctolagus cuniculus
  MISC_FEATURE: 24K1 light chain variable region
MDTRAPTQLLGLLLLWLPGARCADVVMTQTPASVSAVVGGTVTIKCQASQSISSWLSWYQQKLGQPPKLLIYSASTLASGVPSRFKGSGSGADYTLTISDLECADAATYYCQSNTAVHTYNFGGGTEVVVKGDPV

SEQ ID NO: 13 - protein, 15 aa, Homo sapiens
  MISC_FEATURE: 5H2/5K3 epitope 1
SRGPYHPSECCFTYT

SEQ ID NO: 14 - protein, 15 aa, Homo sapiens
  MISC_FEATURE: 5H2/5K3 epitope 2
YETNSQCSKPGIVFI

SEQ ID NO: 15 - protein, 16 aa, Homo sapiens
  MISC_FEATURE: 8H3/8K3 epitope
YYETNSQCSKPGIVFI

SEQ ID NO: 16 - protein, 14 aa, Homo sapiens
  MISC_FEATURE: 9H3/9K2 epitope
SDKWVQDYIKDMKE

SEQ ID NO: 17 - protein, 14 aa, Homo sapiens
  MISC_FEATURE: 14H1/14K1 epitope 1
CCFTYTTYKIPRQR

SEQ ID NO: 18 - protein, 13 aa, Homo sapiens
  MISC_FEATURE: 14H1/14K1 epitope 2
NSQCSKPGIVFIT

SEQ ID NO: 19 - protein, 14 aa, Homo sapiens
  MISC_FEATURE: 15H1/15K3 and 24H1/24K1 epitope sequence
TYKIPRQRIMDYYE

SEQ ID NO: 20 - protein, 93 aa, Homo sapiens
  MISC_FEATURE: Human CCL14 precursor
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 21 - protein, 17 aa, Homo sapiens
  MISC_FEATURE: HCC-3 Domain of CCL14
QTGGKPKVVKIQLKLVG

(19) United States
(12) Patent Application Publication
(10) Pub. No.: US 2022/0356238 A1
(43) Pub. Date: Nov. 10, 2022

(54) ANTIBODIES AND ASSAYS FOR CCL14

(71) Applicant: ASTUTE MEDICAL, INC., SAN DIEGO, CA (US)

(72) Inventors: RAVI A. VIJAYENDRAN, SAN DIEGO, CA (US); HUA WANG, SAN DIEGO, CA (US)

(73) Assignee: ASTUTE MEDICAL, INC., SAN DIEGO, CA (US)

(21) Appl. No.: 17/621,414

(22) PCT Filed: Jul. 1, 2020
(86) PCT No.: PCT/US2020/040520

§ 371 Date: Dec. 21, 2021

Related U.S. Application Data
(60) Provisional application No. 62/869,803, filed on Jul. 2, 2019.

(51) Int. Cl. C07K 16/24; G01N 33/68

(52) U.S. Cl. CPC C07K 16/24; G01N 33/6863; C07K 2317/565; C07K 2317/24

(57) ABSTRACT

The invention provides novel CCL14 antibodies useful in evaluation of renal injuries. In a broad aspect, the present invention provides antibodies which bind CCL14. The provided antibodies can find use in assays to detect CCL14, such as immunoassays with improved clinical performance. In one aspect, the CCL14 antibodies are used in therapeutic methods in which CCL14 binding is desired.

### CROSS REFERENCED APPLICATIONS

This application claims the benefit of U.S. Provisional Application No. 62/869,803, filed on Jul. 2, 2019, which is incorporated herein by reference in its entirety.

### SEQUENCE LISTING

This application contains a sequence listing that has been submitted in ASCII format via EFS-Web and is hereby incorporated by reference in its entirety. Said ASCII copy, created on Jun. 18, 2020, is named 01953_Sequence_Listing_ST25 and is 20.0 KB (20,480 bytes) in size.

### BACKGROUND

Acute renal failure (ARF, also known as acute kidney injury, or AKI) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that ARF complicates about 5% of hospital admissions, 4-15% of cardiopulmonary bypass surgeries, and up to 30% of intensive care admissions.

Although serial measurement of serum creatinine over a period of days is an accepted method of detecting and diagnosing AKI and is considered one of the most important tools to evaluate AKI patients, serum creatinine is generally regarded to have several limitations in the diagnosis, assessment and monitoring of AKI patients. The time period for serum creatinine to rise to values (e.g., a 0.3 mg/dL or 25% rise) considered diagnostic for AKI can be 48 hours or longer depending on the definition used. Since cellular injury in AKI can occur over a period of hours, serum creatinine elevations detected at 48 hours or longer can be a late indicator of injury, and relying on serum creatinine can thus delay diagnosis of AKI. Furthermore, serum creatinine is not a good indicator of the exact kidney status and treatment needs during the most acute phases of AKI when kidney function is changing rapidly. Some patients with AKI will recover fully, some will need dialysis (either short term or long term) and some will have other detrimental outcomes including death, major adverse cardiac events and chronic kidney disease.

Thus, there is a need for better methods to detect and assess acute kidney injury (AKI). Furthermore, there is a need to better identify subjects who are at risk of developing persistent kidney injury or to identify subjects that are likely to recover from AKI. Identifying these subjects can be of vital importance in managing and treating patients with renal injuries.

C-C motif chemokine 14 (CCL14, also known as HCC-1, NCC-2, and SCYA14) is a biomarker shown to be increased in subjects having renal injury. The present invention provides antibodies which bind CCL14. Such antibodies can find use in immunoassays with improved clinical performance, particularly when used in the evaluation of renal injuries, and in therapeutic methods in which CCL14 binding is desired.

### SUMMARY

In a broad aspect, the present invention provides antibodies which bind CCL14. The provided antibodies can find use in assays to detect CCL14, such as immunoassays with improved clinical performance. In one aspect, the CCL14 antibodies are used in therapeutic methods in which CCL14 binding is desired. In another aspect, the CCL14 antibodies are used in the evaluation of renal injuries. Other aspects include methods and kits for detecting CCL14.

In one aspect, an antibody of the present invention binds to an epitope on human CCL14 that comprises all or part of the sequence SRGPYHPSECCFTYT (SEQ ID NO: 13), YETNSQCSKPGIVFI (SEQ ID NO: 14), YYETNSQCSKPGIVFI (SEQ ID NO: 15), SDKWVQDYIKDMKE (SEQ ID NO: 16), CCFTYTTYKIPRQR (SEQ ID NO: 17), NSQCSKPGIVFIT (SEQ ID NO: 18), or TYKIPRQRIMDYYE (SEQ ID NO: 19).

In one aspect, an antibody which competes for binding to human CCL14 with an antibody comprising: three complementarity determining regions (CDRs) of a heavy chain variable region set forth as SEQ ID NO: 1, and three CDRs of a light chain variable region set forth as SEQ ID NO: 2; three CDRs of a heavy chain variable region set forth as SEQ ID NO: 3, and three CDRs of a light chain variable region set forth as SEQ ID NO: 4; three CDRs of a heavy chain variable region set forth as SEQ ID NO: 5, and three CDRs of a light chain variable region set forth as SEQ ID NO: 6; three CDRs of a heavy chain variable region set forth as SEQ ID NO: 7, and three CDRs of a light chain variable region set forth as SEQ ID NO: 8; three CDRs of a heavy chain variable region set forth as SEQ ID NO: 9, and three CDRs of a light chain variable region set forth as SEQ ID NO: 10; or three CDRs of a heavy chain variable region set forth as SEQ ID NO: 11, and three CDRs of a light chain variable region set forth as SEQ ID NO: 12, is provided.

In another aspect, the present invention relates to antibodies comprising: three complementarity determining regions (CDRs) of a heavy chain variable region set forth as SEQ ID NO: 1, and three CDRs of a light chain variable region set forth as SEQ ID NO: 2; three CDRs of a heavy chain variable region set forth as SEQ ID NO: 3, and three CDRs of a light chain variable region set forth as SEQ ID NO: 4; three CDRs of a heavy chain variable region set forth as SEQ ID NO: 5, and three CDRs of a light chain variable region set forth as SEQ ID NO: 6; three CDRs of a heavy chain variable region set forth as SEQ ID NO: 7, and three CDRs of a light chain variable region set forth as SEQ ID NO: 8; three CDRs of a heavy chain variable region set forth as SEQ ID NO: 9, and three CDRs of a light chain variable region set forth as SEQ ID NO: 10; or three CDRs of a heavy chain variable region set forth as SEQ ID NO: 11, and three CDRs of a light chain variable region set forth as SEQ ID NO: 12.

In another aspect, the present invention relates to antibodies or antigen binding fragments thereof that binds to human CCL14, wherein the antibody or antigen binding fragment comprises:

(i) a heavy chain variable region comprising
a CDR1, CDR2, and CDR3 sequence from SEQ ID NO: 1,
a CDR1, CDR2, and CDR3 sequence from SEQ ID NO: 3,
a CDR1, CDR2, and CDR3 sequence from SEQ ID NO: 5,
a CDR1, CDR2, and CDR3 sequence from SEQ ID NO: 7,
a CDR1, CDR2, and CDR3 sequence from SEQ ID NO: 9, or
a CDR1, CDR2, and CDR3 sequence from SEQ ID NO: 11;
and

(ii) a light chain variable region comprising
a CDR1, CDR2, and CDR3 sequence from SEQ ID NO: 2,
a CDR1, CDR2, and CDR3 sequence from SEQ ID NO: 4,
a CDR1, CDR2, and CDR3 sequence from SEQ ID NO: 6,
a CDR1, CDR2, and CDR3 sequence from SEQ ID NO: 8,
a CDR1, CDR2, and CDR3 sequence from SEQ ID NO: 10, or
a CDR1, CDR2, and CDR3 sequence from SEQ ID NO: 12.

In certain aspects, the antibody or antigen binding fragment comprises one the following heavy chain CDR/light chain CDR pairs:

a heavy chain variable region comprising a CDR1, CDR2, and CDR3 sequence from SEQ ID NO: 1, and a light chain variable region comprising a CDR1, CDR2, and CDR3 sequence from SEQ ID NO: 2 (5H2/5K3),
a heavy chain variable region comprising a CDR1, CDR2, and CDR3 sequence from SEQ ID NO: 3, and a light chain variable region comprising a CDR1, CDR2, and CDR3 sequence from SEQ ID NO: 4 (8H3/8K3),
a heavy chain variable region comprising a CDR1, CDR2, and CDR3 sequence from SEQ ID NO: 5, and a light chain variable region comprising a CDR1, CDR2, and CDR3 sequence from SEQ ID NO: 6 (9H3/9K2),
a heavy chain variable region comprising a CDR1, CDR2, and CDR3 sequence from SEQ ID NO: 7, and a light chain variable region comprising a CDR1, CDR2, and CDR3 sequence from SEQ ID NO: 8 (14H1/14K1),
a heavy chain variable region comprising a CDR1, CDR2, and CDR3 sequence from SEQ ID NO: 9, and a light chain variable region comprising a CDR1, CDR2, and CDR3 sequence from SEQ ID NO: 10 (15H1/15K3), or
a heavy chain variable region comprising a CDR1, CDR2, and CDR3 sequence from SEQ ID NO: 11, and a light chain variable region comprising a CDR1, CDR2, and CDR3 sequence from SEQ ID NO: 12 (24H1/24K1).

In certain aspects, the antibody comprises: residues 27-38 of SEQ ID NO: 1 for CDR-H1, residues 56-65 of SEQ ID NO: 1 for CDR-H2, residues 105-117 of SEQ ID NO: 1 for CDR-H3, residues 27-38 of SEQ ID NO: 2 for CDR-L1, residues 56-65 of SEQ ID NO: 2 for CDR-L2 and residues 105-117 of SEQ ID NO: 2 for CDR-L3; residues 27-38 of SEQ ID NO: 3 for CDR-H1, residues 56-65 of SEQ ID NO: 3 for CDR-H2, residues 105-117 of SEQ ID NO: 3 for CDR-H3, residues 27-38 of SEQ ID NO: 4 for CDR-L1, residues 56-65 of SEQ ID NO: 4 for CDR-L2 and residues 105-117 of SEQ ID NO: 4 for CDR-L3; residues 27-38 of SEQ ID NO: 5 for CDR-H1, residues 56-65 of SEQ ID NO: 5 for CDR-H2, residues 105-117 of SEQ ID NO: 5 for CDR-H3, residues 27-38 of SEQ ID NO: 6 for CDR-L1, residues 56-65 of SEQ ID NO: 6 for CDR-L2 and residues 105-117 of SEQ ID NO: 6 for CDR-L3; residues 27-38 of SEQ ID NO: 7 for CDR-H1, residues 56-65 of SEQ ID NO: 7 for CDR-H2, residues 105-117 of SEQ ID NO: 7 for CDR-H3, residues 27-38 of SEQ ID NO: 8 for CDR-L1, residues 56-65 of SEQ ID NO: 8 for CDR-L2 and residues 105-117 of SEQ ID NO: 8 for CDR-L3; residues 27-38 of SEQ ID NO: 9 for CDR-H1, residues 56-65 of SEQ ID NO: 9 for CDR-H2, residues 105-117 of SEQ ID NO: 9 for CDR-H3, residues 27-38 of SEQ ID NO: 10 for CDR-L1, residues 56-65 of SEQ ID NO: 10 for CDR-L2 and residues 105-117 of SEQ ID NO: 10 for CDR-L3; or residues 27-38 of SEQ ID NO: 11 for CDR-H1, residues 56-65 of SEQ ID NO: 11 for CDR-H2, residues 105-117 of SEQ ID NO: 11 for CDR-H3, residues 27-38 of SEQ ID NO: 12 for CDR-L1, residues 56-65 of SEQ ID NO: 12 for CDR-L2 and residues 105-117 of SEQ ID NO: 12 for CDR-L3, wherein the residues are numbered according to Lefranc.

In certain aspects, the antibody comprises: residues 31-35 of SEQ ID NO: 1 for CDR-H1, residues 50-65 of SEQ ID NO: 1 for CDR-H2 residues 95-102 of SEQ ID NO: 1 for CDR-H3, residues 24-34 of SEQ ID NO: 2 for CDR-L1, residues 50-56 of SEQ ID NO: 2 for CDR-L2, and residues 89-97 of SEQ ID NO: 2 for CDR-L3; residues 31-35 of SEQ ID NO: 3 for CDR-H1, residues 50-65 of SEQ ID NO: 3 for CDR-H2 residues 95-102 of SEQ ID NO: 3 for CDR-H3, residues 24-34 of SEQ ID NO: 4 for CDR-L1, residues 50-56 of SEQ ID NO: 4 for CDR-L2, and residues 89-97 of SEQ ID NO: 4 for CDR-L3; residues 31-35 of SEQ ID NO: 5 for CDR-H1, residues 50-65 of SEQ ID NO: 5 for CDR-H2 residues 95-102 of SEQ ID NO: 5 for CDR-H3, residues 24-34 of SEQ ID NO: 6 for CDR-L1, residues 50-56 of SEQ ID NO: 6 for CDR-L2, and residues 89-97 of SEQ ID NO: 6 for CDR-L3; residues 31-35 of SEQ ID NO: 7 for CDR-H1, residues 50-65 of SEQ ID NO: 7 for CDR-H2 residues 95-102 of SEQ ID NO: 7 for CDR-H3, residues 24-34 of SEQ ID NO: 8 for CDR-L1, residues 50-56 of SEQ ID NO: 8 for CDR-L2, and residues 89-97 of SEQ ID NO: 8 for CDR-L3; residues 31-35 of SEQ ID NO: 9 for CDR-H1, residues 50-65 of SEQ ID NO: 9 for CDR-H2 residues 95-102 of SEQ ID NO: 9 for CDR-H3, residues 24-34 of SEQ ID NO: 10 for CDR-L1, residues 50-56 of SEQ ID NO: 10 for CDR-L2, and residues 89-97 of SEQ ID NO: 10 for CDR-L3; or residues 31-35 of SEQ ID NO: 11 for CDR-H1, residues 50-65 of SEQ ID NO: 11 for CDR-H2 residues 95-102 of SEQ ID NO: 11 for CDR-H3, residues 24-34 of SEQ ID NO: 12 for CDR-L1, residues 50-56 of SEQ ID NO: 12 for CDR-L2, and residues 89-97 of SEQ ID NO: 12 for CDR-L3, wherein the residues are numbered according to Kabat.

In certain aspects, the antibody comprises: residues 26-32 of SEQ ID NO: 1 for CDR-H1, residues 52-56 of SEQ ID NO: 1 for CDR-H2 residues 95-102 of SEQ ID NO: 1 for CDR-H3, residues 24-34 of SEQ ID NO: 2 for CDR-L1, residues 50-56 of SEQ ID NO: 2 for CDR-L2, and residues 89-97 of SEQ ID NO: 2 for CDR-L3; residues 26-32 of SEQ ID NO: 3 for CDR-H1, residues 52-56 of SEQ ID NO: 3 for CDR-H2 residues 95-102 of SEQ ID NO: 3 for CDR-H3, residues 24-34 of SEQ ID NO: 4 for CDR-L1, residues 50-56 of SEQ ID NO: 4 for CDR-L2, and residues 89-97 of SEQ ID NO: 4 for CDR-L3; residues 26-32 of SEQ ID NO: 5 for CDR-H1, residues 52-56 of SEQ ID NO: 5 for CDR-H2 residues 95-102 of SEQ ID NO: 5 for CDR-H3, residues 24-34 of SEQ ID NO: 6 for CDR-L1, residues 50-56 of SEQ ID NO: 6 for CDR-L2, and residues 89-97 of SEQ ID NO: 6 for CDR-L3; residues 26-32 of SEQ ID NO: 7 for CDR-H1, residues 52-56 of SEQ ID NO: 7 for CDR-H2 residues 95-102 of SEQ ID NO: 7 for CDR-H3, residues 24-34 of SEQ ID NO: 8 for CDR-L1, residues 50-56 of SEQ ID NO: 8 for CDR-L2, and residues 89-97 of SEQ ID NO: 8 for CDR-L3; residues 26-32 of SEQ ID NO: 9 for CDR-H1, residues 52-56 of SEQ ID NO: 9 for CDR-H2 residues 95-102 of SEQ ID NO: 9 for CDR-H3, residues 24-34 of SEQ ID NO: 10 for CDR-L1, residues 50-56 of SEQ ID NO: 10 for CDR-L2, and residues 89-97 of SEQ ID NO: 10 for CDR-L3; or residues 26-32 of SEQ ID NO: 11 for CDR-H1, residues 52-56 of SEQ ID NO: 11 for CDR-H2 residues 95-102 of SEQ ID NO: 11 for CDR-H3, residues 24-34 of SEQ ID NO: 12 for CDR-L1, residues 50-56 of SEQ ID NO: 12 for CDR-L2, and residues 89-97 of SEQ ID NO: 12 for CDR-L3, wherein the residues are numbered according to Chothia.

In certain aspects, the antibody comprises: residues 30-35 of SEQ ID NO: 1 for CDR-H1, residues 47-58 of SEQ ID NO: 1 for CDR-H2 residues 93-101 of SEQ ID NO: 1 for CDR-H3, residues 30-36 of SEQ ID NO: 2 for CDR-L1, residues 46-55 of SEQ ID NO: 2 for CDR-L2, and residues 89-96 of SEQ ID NO: 2 for CDR-L3; residues 30-35 of SEQ ID NO: 3 for CDR-H1, residues 47-58 of SEQ ID NO: 3 for CDR-H2 residues 93-101 of SEQ ID NO: 3 for CDR-H3, residues 30-36 of SEQ ID NO: 4 for CDR-L1, residues 46-55 of SEQ ID NO: 4 for CDR-L2, and residues 89-96 of SEQ ID NO: 4 for CDR-L3; residues 30-35 of SEQ ID NO: 5 for CDR-H1, residues 47-58 of SEQ ID NO: 5 for CDR-H2 residues 93-101 of SEQ ID NO: 5 for CDR-H3, residues 30-36 of SEQ ID NO: 6 for CDR-L1, residues 46-55 of SEQ ID NO: 6 for CDR-L2, and residues 89-96 of SEQ ID NO: 6 for CDR-L3; residues 30-35 of SEQ ID NO: 7 for CDR-H1, residues 47-58 of SEQ ID NO: 7 for CDR-H2 residues 93-101 of SEQ ID NO: 7 for CDR-H3, residues 30-36 of SEQ ID NO: 8 for CDR-L1, residues 46-55 of SEQ ID NO: 8 for CDR-L2, and residues 89-96 of SEQ ID NO: 8 for CDR-L3; residues 30-35 of SEQ ID NO: 9 for CDR-H1, residues 47-58 of SEQ ID NO: 9 for CDR-H2 residues 93-101 of SEQ ID NO: 9 for CDR-H3, residues 30-36 of SEQ ID NO: 10 for CDR-L1, residues 46-55 of SEQ ID NO: 10 for CDR-L2, and residues 89-96 of SEQ ID NO: 10 for CDR-L3; or residues 30-35 of SEQ ID NO: 11 for CDR-H1, residues 47-58 of SEQ ID NO: 11 for CDR-H2 residues 93-101 of SEQ ID NO: 11 for CDR-H3, residues 30-36 of SEQ ID NO: 12 for CDR-L1, residues 46-55 of SEQ ID NO: 12 for CDR-L2, and residues 89-96 of SEQ ID NO: 12 for CDR-L3, wherein the residues are numbered according to MacCallum.

In certain aspects, the antibody comprises: a heavy chain variable region comprising an amino acid sequence set forth as SEQ ID NO: 1 and a light chain variable region comprising an amino acid sequence set forth as SEQ ID NO: 2; a heavy chain variable region comprising an amino acid sequence set forth as SEQ ID NO: 3 and a light chain variable region comprising an amino acid sequence set forth as SEQ ID NO: 4; a heavy chain variable region comprising an amino acid sequence set forth as SEQ ID NO: 5 and a light chain variable region comprising an amino acid sequence set forth as SEQ ID NO: 6; a heavy chain variable region comprising an amino acid sequence set forth as SEQ ID NO: 7 and a light chain variable region comprising an amino acid sequence set forth as SEQ ID NO: 8; a heavy chain variable region comprising an amino acid sequence set forth as SEQ ID NO: 9 and a light chain variable region comprising an amino acid sequence set forth as SEQ ID NO: 10; or a heavy chain variable region comprising an amino acid sequence set forth as SEQ ID NO: 11 and a light chain variable region comprising an amino acid sequence set forth as SEQ ID NO: 12.

In certain aspects, the antibody or antigen binding fragment comprises:

(i) a heavy chain variable region selected from the group consisting of SEQ ID NO: 1, 3, 5, 7, 9, and 11, or a corresponding heavy chain variable region having at least 90% sequence similarity to the framework region thereof; and (ii) a light chain variable region selected from the group consisting of SEQ ID NO: 2, 4, 6, 8, 10, or 12, or a corresponding light chain variable region having at least 90% sequence similarity to the framework region thereof.

In certain aspects, the antibody or antigen binding fragment comprises one the following heavy chain/light chain pairs: a heavy chain variable region of SEQ ID NO: 1 or a corresponding heavy chain variable region having at least 90% sequence similarity to the framework region thereof, and a light chain variable region of SEQ ID NO: 2 a corresponding light chain variable region having at least 90% sequence similarity to the framework region thereof (5H3/5K3).

a heavy chain variable region of SEQ ID NO: 3 or a corresponding heavy chain variable region having at least 90% sequence similarity to the framework region thereof, and a light chain variable region of SEQ ID NO: 4 a corresponding light chain variable region having at least 90% sequence similarity to the framework region thereof (8H3/8K3),
a heavy chain variable region of SEQ ID NO: 5 or a corresponding heavy chain variable region having at least 90% sequence similarity to the framework region thereof, and a light chain variable region of SEQ ID NO: 6 a corresponding light chain variable region having at least 90% sequence similarity to the framework region thereof (9H3/9K2),
a heavy chain variable region of SEQ ID NO: 7 or a corresponding heavy chain variable region having at least 90% sequence similarity to the framework region thereof, and a light chain variable region of SEQ ID NO: 8 a corresponding light chain variable region having at least 90% sequence similarity to the framework region thereof (14H1/14K1),
a heavy chain variable region of SEQ ID NO: 9 or a corresponding heavy chain variable region having at least 90% sequence similarity to the framework region thereof, and a light chain variable region of SEQ ID NO: 10 a corresponding light chain variable region having at least 90% sequence similarity to the framework region thereof (15H1/15K3), or
a heavy chain variable region of SEQ ID NO: 11 or a corresponding heavy chain variable region having at least 90% sequence similarity to the framework region thereof, and a light chain variable region of SEQ ID NO: 12 a corresponding light chain variable region having at least 90% sequence similarity to the framework region thereof (24H1/24K1).

Within the description of the present invention, at least 90% sequence similarity should be understood to include at least 95%, and more preferably at least 99% sequence similarity. In this context, “sequence similarity” is based on the extent of identity combined with the extent of conservative changes. The percentage of “sequence similarity” is the percentage of amino acids or nucleotides which is either identical or conservatively changed viz. “sequence similarity”=percent sequence identity)+percent conservative changes). Thus, for the purpose of this invention “conservative changes” and “identity” are considered to be species of the broader term “similarity”. Thus, whenever the term sequence “similarity” is used it embraces sequence “identity” and “conservative changes”. According to certain aspects the conservative changes are disregarded and the percent sequence similarity refers to percent sequence identity. In certain aspects, the changes in a sequence permitted by the referenced percent sequence identity are all or nearly all conservative changes; that is, when a sequence is 90% identical, the remaining 10% are all or nearly all conservative changes. The term “nearly all” in this context refers to at least 75% of the permitted sequence changes are conservative changes, more preferably at least 85%, still more preferably at least 90%, and most preferably at least 95%.

Antibodies for use in the claimed methods may be obtained from a variety of species. For example, the antibodies of the present invention may comprise immunoglobulin sequences which are rabbit, mouse, rat, guinea pig, chicken, goat, sheep, donkey, human, llama or camelid sequences, or combinations of such sequences (so-called chimeric antibodies).

The antibodies of the invention may be monoclonal or polyclonal or antigen binding fragments thereof. In certain aspects, the antibodies of the invention are humanized. In other aspects, the antibodies of the invention are antigen binding fragments, such as an F(ab) fragment, F(ab′)2 fragment, Fv fragment, Fd fragment, or dAb fragment.

Nucleic acids encoding the antibodies of the present invention are also provided. In certain aspects, vectors comprising the nucleic acids are provided. In certain aspects, the nucleic acid encodes an amino acid heavy chain variable region and/or an amino acid light chain variable region of an antibody of the invention. In other aspects host cells comprising the nucleic acids or vectors of the invention are provided.

Antibodies for use in the present invention may be identified by their performance in immunoassays, and then further characterized by epitope mapping in order to understand the epitopes which are relevant to that performance. Preferred are rabbit antibodies or humanized versions derived from rabbit antibodies.

Such antibodies may be conjugated to a signal development element or immobilized on a solid support. In addition, such antibodies may be used in a number of competitive and sandwich assay formats. In an example of a sandwich assay, a first antibody (detectably labeled) and a second antibody (immobilized at a predetermined zone of a test device) form a sandwich complex with CCL14 in the sample at a predetermined zone of a test device. In sandwich assays, the first and second antibodies can be the same (particularly when polyclonal antibodies are used) or different. Thus, the antibodies of the invention are used in sandwich pairs, or are used individually with another binding entity which is not a monoclonal antibody such as a polyclonal antibody or an aptamer. In other aspects, the antibodies of the invention are used in assays or as a sandwich pair (e.g., a sandwich assay) with other known CCL14 antibodies.

In certain aspects, kits comprising an antibody of the present invention are provided.

The antibodies of the present invention can be used as reagents in test kits for detecting CCL14 in samples, including, for example, a body fluid sample. In some aspects the kits comprise a first antibody, and a second antibody which specifically binds human CCL14, wherein the first antibody and the second antibody form a sandwich complex with human CCL14. In certain aspects, the second antibody or antigen binding fragment is a different antibody from the first antibody or antigen binding fragment. In other aspects, one or both of the first antibody and second antibody is a monoclonal antibody, a polyclonal antibody, a humanized antibody, an F(ab) fragment, an F(ab′)2 fragment, an Fv fragment, an Fd fragment, or a dAb fragment; or an antigen binding fragment thereof. Such a test kit may, for example, comprise a disposable test device configured to generate a detectable signal related to the present or amount of human CCL14 in a body fluid sample. Alternatively, such a test kit may be formulated for performing an assay in a clinical analyzer which does not utilize a disposable test device. Preferably, the test kit is an in vitro diagnostic. The term “in vitro diagnostic” as used herein refers to a medical device which is a reagent, reagent product, calibrator, control material, kit, instrument, apparatus, equipment, or system, whether used alone or in combination, intended by the manufacturer to be used in vitro for the examination of specimens, including blood and tissue donations, derived from the human body, solely or principally for the purpose of providing information concerning a physiological or pathological state, or concerning a congenital abnormality, or to determine the safety and compatibility with potential recipients, or to monitor therapeutic measures.

In certain aspects, a kit is provided comprising any of the various CCL14 antibodies of the present invention and instructions for performing an immunoassay for CCL14. In one aspect, the immunoassay is a competitive immunoassay.

In certain aspects, the immunoassay is performed in a lateral flow format. Lateral flow tests are a form of immunoassay in which the test sample flows in a chromatographic fashion along a bibulous or non-bibulous porous solid substrate. Lateral flow tests can operate as either competitive or sandwich format assays. Preferred lateral flow devices are disposable, single use test devices. A sample is applied to the test device at an application zone and transits the substrate, where it encounters lines or zones which have been pretreated with an antibody or antigen. The term “test zone” as used herein refers to a discrete location on a lateral flow test strip which is interrogated in order to generate a signal related to the presence or amount of an analyte of interest. The detectable signal may be read visually or obtained by inserting the disposable test device into an analytical instrument such as a reflectometer, a fluorometer, or a transmission photometer. This list is not meant to be limiting. Sample may be applied without pretreatment to the application zone, or may be premixed with one or more assay reagents prior to application. In the latter case, the antibody may be provided in a separate container from the disposable test device.

An antibody of the present invention may be diffusively immobilized to a surface within a test device, such that the antibody dissolves into a sample when the sample contacts the surface. In a sandwich assay format, this diffusively bound antibody may bind to its cognate antigen in the sample, and then be immobilized at a detection zone when the antigen is bound by a second antibody non-diffusively bound at the detection zone. In a competitive format, its cognate antigen in the sample may compete for binding to the non-diffusively bound antibody with a labeled antigen provided as an assay reagent. In some aspects, the test device is a disposable test device.

A kit of the invention can further comprise a calibration curve to relate the detectable signal to a concentration of CCL14. By way of example, a calibration curve may be provided on an electronic memory device which is read by the analytical instrument which receives the test device, such as a ROM chip, a flash drive, an RFID tag, etc. Alternatively, the calibration curve may be provided on an encoded label which is read optically, such as a 2-D bar code, or transmitted via a network connection. The analytical instrument can then use this calibration curve to relate a detectable signal from an assay into a CCL14 concentration. In some aspects, the test device is a disposable test device. In addition, the kit may provide reagents useful for generating a calibration curve. In some aspects, the reagents include, for example, a CCL14 protein, such as a human CCL14 protein. By way of example, the calibration curve may be generated by preparing various known concentrations of the CCL14 protein.

In certain aspects, an assay method performed using one or more antibodies of the present invention provides a signal related to the presence or amount of human CCL14 in a body fluid sample, wherein the minimum detectable concentration of CCL14 in the assay method is 10 ng/mL or less, more preferably 1 ng/mL or less, and most preferably 0.1 ng/mL or less.

In related aspects, the present invention provides methods for determining the presence or amount of human CCL14 in a sample, including, for example, a body fluid sample, comprising:

performing an immunoassay on the sample with a first antibody and a second antibody which together form a sandwich complex with human CCL14, wherein the immunoassay provides a detectable signal that is related to a presence or an amount of human CCL14 in the sample bound in the sandwich complex; and relating the detectable signal to the presence or amount of human CCL14 in the sample. In certain aspects, one or both of the first antibody and second antibody is a monoclonal antibody, a polyclonal antibody, a humanized antibody, an F(ab) fragment, an F(ab′)₂ fragment, an Fv fragment, an Fd fragment, or a dAb fragment; or an antigen binding fragment thereof. Preferably, the minimum detectable concentration of CCL14 in the immunoassay is 10 ng/mL or less, more preferably 1 ng/mL or less, and most preferably 0.1 ng/mL or less.

In particularly preferred aspects, the immunoassay is a sandwich immunoassay, in which each of the first and second antibodies are an antibody (which may be an antigen binding fragment) of the present invention. By way of example, the first antibody in the sandwich pair comprises one of the following heavy chain CDR/light chain CDR pairs, and the second antibody in the sandwich pair comprises a different one of the following heavy chain CDR/light chain CDR pairs:

a heavy chain variable region comprising a CDR1, CDR2, and CDR3 sequence from SEQ ID NO: 1, and a light chain variable region comprising a CDR1, CDR2, and CDR3 sequence from SEQ ID NO: 2 (5H2/5K3),

a heavy chain variable region comprising a CDR1, CDR2, and CDR3 sequence from SEQ ID NO: 3, and a light chain variable region comprising a CDR1, CDR2, and CDR3 sequence from SEQ ID NO: 4 (8H3/8K3),

a heavy chain variable region comprising a CDR1, CDR2, and CDR3 sequence from SEQ ID NO: 5, and a light chain variable region comprising a CDR1, CDR2, and CDR3 sequence from SEQ ID NO: 6 (9H3/9K2),
a heavy chain variable region comprising a CDR1, CDR2, and CDR3 sequence from SEQ ID NO: 7, and a light chain variable region comprising a CDR1, CDR2, and CDR3 sequence from SEQ ID NO: 8 (14H1/14K1),
a heavy chain variable region comprising a CDR1, CDR2, and CDR3 sequence from SEQ ID NO: 9, and a light chain variable region comprising a CDR1, CDR2, and CDR3 sequence from SEQ ID NO: 10 (15H1/15K3), or
a heavy chain variable region comprising a CDR1, CDR2, and CDR3 sequence from SEQ ID NO: 11, and a light chain variable region comprising a CDR1, CDR2, and CDR3 sequence from SEQ ID NO: 12 (24H1/24K1).

In certain aspects, the first antibody in the sandwich pair comprises one of the following heavy chain CDR/light chain CDR pairs, and the second antibody in the sandwich pair comprises a different one of the following heavy chain/light chain pairs: a heavy chain variable region of SEQ ID NO: 1 or a corresponding heavy chain variable region having at least 90% sequence similarity to the framework region thereof, and a light chain variable region of SEQ ID NO: 2 a corresponding light chain variable region having at least 90% sequence similarity to the framework region thereof (5H2/5K3).

a heavy chain variable region of SEQ ID NO: 3 or a corresponding heavy chain variable region having at least 90% sequence similarity to the framework region thereof, and a light chain variable region of SEQ ID NO: 4 a corresponding light chain variable region having at least 90% sequence similarity to the framework region thereof (8H3/8K3),
a heavy chain variable region of SEQ ID NO: 5 or a corresponding heavy chain variable region having at least 90% sequence similarity to the framework region thereof, and a light chain variable region of SEQ ID NO: 6 a corresponding light chain variable region having at least 90% sequence similarity to the framework region thereof (9H3/9K2),
a heavy chain variable region of SEQ ID NO: 7 or a corresponding heavy chain variable region having at least 90% sequence similarity to the framework region thereof, and a light chain variable region of SEQ ID NO: 8 a corresponding light chain variable region having at least 90% sequence similarity to the framework region thereof (14H1/14K1),
a heavy chain variable region of SEQ ID NO: 9 or a corresponding heavy chain variable region having at least 90% sequence similarity to the framework region thereof, and a light chain variable region of SEQ ID NO: 10 a corresponding light chain variable region having at least 90% sequence similarity to the framework region thereof (15H1/15K3), or
a heavy chain variable region of SEQ ID NO: 11 or a corresponding heavy chain variable region having at least 90% sequence similarity to the framework region thereof, and a light chain variable region of SEQ ID NO: 12 a corresponding light chain variable region having at least 90% sequence similarity to the framework region thereof (24H1/24K1).

In certain aspects, the present invention provides methods for determining the presence or amount of human CCL14 in a body fluid sample, comprising performing a competitive immunoassay on the body fluid sample with an antibody of the present invention which binds human CCL14, wherein the competitive immunoassay provides a detectable signal, and relating the detectable signal to the presence or amount of human CCL14 in the body fluid sample.

In related aspects, the present invention relates to antibodies that bind to epitopes of an antibody of the present invention, or that compete for binding to CCL14 with an antibody of the present invention. As described herein, such antibodies may find use in kits, in antibody pairs, in methods, and in assay devices.

In preferred aspects, a monoclonal antibody of the present invention binds to an epitope on human CCL14 that comprises all or part of the sequence SRGPYHPSECCFTYT (SEQ ID NO: 13), YETNSQCSKPGIVFI (SEQ ID NO: 14), YYETNSQCSKPGIVFI (SEQ ID NO: 15), SDKWVQDYIKDMKE (SEQ ID NO: 16), CCFTYTTYKIPRQR (SEQ ID NO: 17), NSQCSKPGIVFIT (SEQ ID NO: 18), or TYKIPRQRIMDYYE (SEQ ID NO: 19), and is most preferably a rabbit monoclonal antibody.

In certain aspects, the present invention further comprises a second monoclonal antibody or antigen binding fragment which specifically binds human CCL14 and that binds to an epitope on human CCL14 that comprises all or part of the sequence SRGPYHPSECCFTYT (SEQ ID NO: 13), YETNSQCSKPGIVFI (SEQ ID NO: 14), YYETNSQCSKPGIVFI (SEQ ID NO: 15), SDKWVQDYIKDMKE (SEQ ID NO: 16), CCFTYTTYKIPRQR (SEQ ID NO: 17), NSQCSKPGIVFIT (SEQ ID NO: 18), or TYKIPRQRIMDYYE (SEQ ID NO: 19), wherein the monoclonal antibody and the second antibody form a sandwich complex with human CCL14.

Preferred assay methods comprise performing an immunoassay that detects human CCL14. Such immunoassays may comprise contacting said body fluid sample with an antibody that detects the marker, and detecting binding to that antibody. While the present invention is generally described in terms of immunoassays, other binding entities (e.g., aptamers) which are not based on an immunoglobulin scaffold may be used in lieu of antibodies in such methods. Preferably, the body fluid sample is selected from the group consisting of urine, saliva, blood, serum, and plasma, and most preferably urine.

The details of one or more aspects of the disclosure are set forth in the accompanying drawings and the description below. Other features, objects, and advantages of the disclosure will be apparent from the description and drawings, and from the claims.

### BRIEF DESCRIPTION OF THE FIGURES

FIG. 1 shows a variable region alignment of the protein sequence for 5H2, 8H3, 9H3, 14H1, 15H1 and 24H1 rabbit IgG heavy chains of the invention. The three complementarity determining regions (CDR) are indicated.

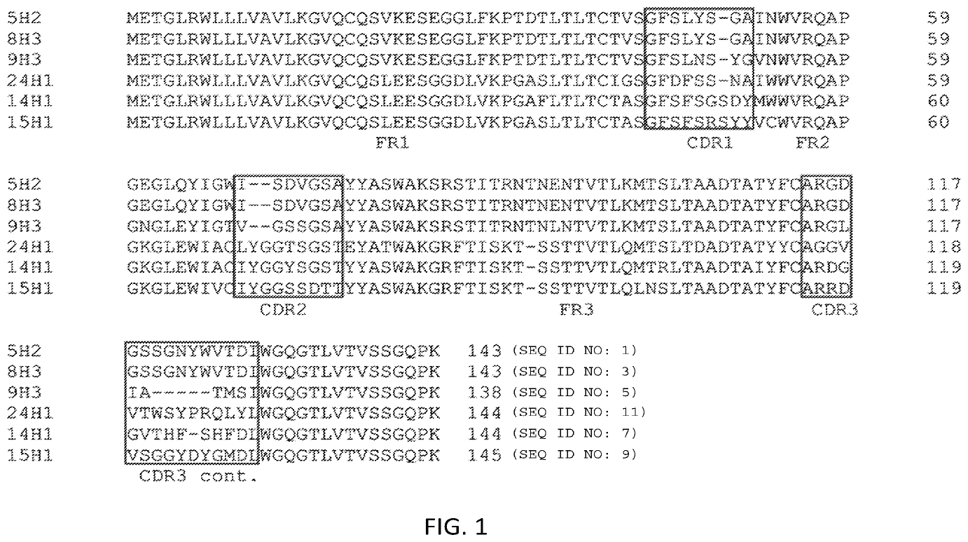

FIG. 2 shows a variable region alignment of the protein sequence for 5K3, 8K3, 9K2, 14K1, 15K3 and 24K1 rabbit light chain. The three complementarity determining regions (CDR) are indicated.

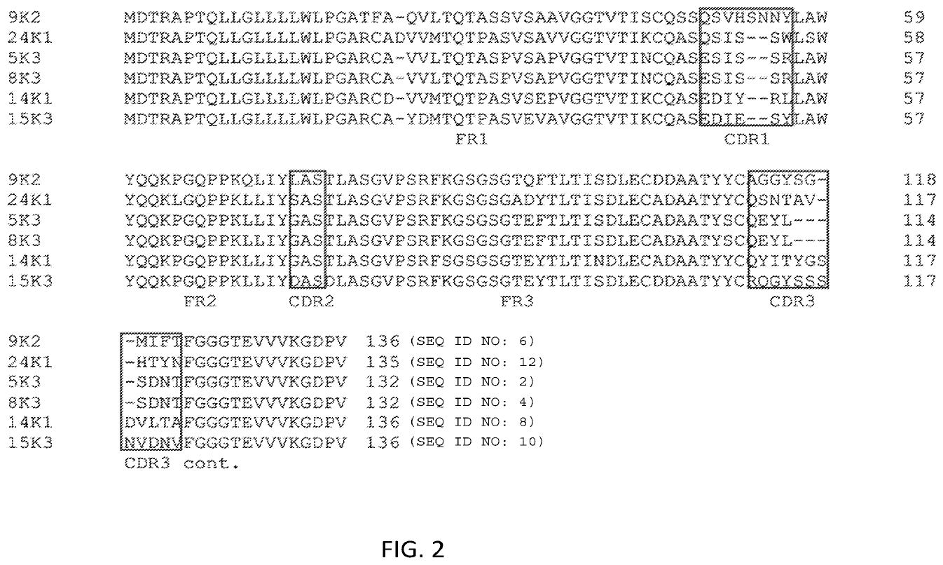

### DETAILED DESCRIPTION

C-C motif chemokine 14 has been found to correlate with renal injury. See, for example, International publication numbers WO 2016/064877 and WO 2018/132702 and U.S. publication number 2018/0209990, which are incorporated by reference herein in their entirety.

As used herein, the terms “C-C motif chemokine 14” and “CCL14” refer to one or more polypeptides present in a sample obtained from a subject, including, for example, a body fluid sample, that are derived from the CCL14 precursor (human precursor: Swiss-Prot Q16627 (SEQ ID NO: 20)).

```
                10 20 30 40
MKISVAAIPF FLLITIALGT KTESSSRGPY HPSECCFTYT
                50 60 70 80
TYKIPRQRIM DYYETNSQCS KPGIVFITKR GHSVCTNPSD
                90
KWVQDYIKDM KEN
```

The following domains have been identified in CCL14:

| Residues | Length | Domain ID |
|---|---|---|
| 1-19 | 19 | Signal peptide |
| 20-93 | 74 | CCL14 |
| 22-93 | 72 | HCC-l(3-74) |
| 23-93 | 71 | HCC-1(4-74) |
| 28-93 | 66 | HCC-1(9-74) |
| 27 | R → QTGGKPKVVKIQLKLVG | HCC-3 |
| | (SEQ ID NO: 21) | |

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte is measured in a sample. Such a sample is obtained from a subject, or is obtained from biological materials intended to be provided to the subject. For example, a sample may be obtained from a kidney being evaluated for possible transplantation into a subject, and an analyte measurement used to evaluate the kidney for preexisting damage. In certain aspects, the sample is a tissue sample. In other aspects, the sample is a body fluid sample.

The term “body fluid sample” as used herein refers to a sample of bodily fluid obtained for the purpose of diagnosis, prognosis, classification or evaluation of a subject of interest, such as a patient or transplant donor. In certain aspects, such a sample is obtained for the purpose of determining the outcome of an ongoing condition or the effect of a treatment regimen on a condition. Preferred body fluid samples include blood, serum, plasma, cerebrospinal fluid, urine, saliva, sputum, and pleural effusions. In addition, one of skill in the art would realize that certain body fluid samples would be more readily analyzed following a fractionation or purification procedure, for example, separation of whole blood into serum or plasma components.

The CCL14 antibodies provided in the present invention find use in evaluating renal function in a subject, including use in assays for the diagnosis and prognosis of renal injury, such as acute kidney injury and acute renal failure. “Acute renal failure” or “ARF” is an abrupt (within 14 days, preferably within 7 days, more preferably within 72 hours, and still more preferably within 48 hours) reduction in kidney function identified by an absolute increase in serum creatinine of greater than or equal to 0.3 mg/dl (≥26.4 μmol/l), a percentage increase in serum creatinine of greater than or equal to 50% (1.5-fold from baseline), or a reduction in urine output (documented oliguria of less than 0.5 ml/kg per hour for at least 6 hours). This term is synonymous with “acute kidney injury” or “AKI.”

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, for a kidney injury marker of the present invention, optionally together with other clinical characteristics, to arrive at a diagnosis (that is, the occurrence or nonoccurrence) of an acute renal injury or acute renal failure (ARF) for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening renal function, future ARF, or death) is referred to as being “indicative of an increased likelihood” of an adverse outcome in a patient. In the case of the present invention, the results of an assay for CCL14 can be used for monitoring renal function in a subject who has suffered from an injury to renal function or reduced renal function, including the likelihood for future persistence of acute kidney injury, the likelihood for progression to ARF, the likelihood that a subject will require renal replacement therapy, the likelihood for progression to end stage renal disease, the likelihood for progression to chronic renal failure, the likelihood for future improvement of renal function or the likelihood of future recovery from ARF.

In such prognostic risk stratification, preferably the likelihood or risk assigned is that an event of interest is more or less likely to occur within 180 days of the time at which the body fluid sample is obtained from the subject. In particularly preferred embodiments, the likelihood or risk assigned relates to an event of interest occurring within a shorter time period such as 18 months, 120 days, 90 days, 60 days, 45 days, 30 days, 21 days, 14 days, 7 days, 5 days, 96 hours, 72 hours, 48 hours, 36 hours, 24 hours, 12 hours, or less. A risk at 0 hours of the time at which the body fluid sample is obtained from the subject is equivalent to diagnosis of a current condition.

In still other aspects, the CCL14 antibodies provided in the present invention are used in methods for evaluating and/or monitoring a renal injury in a subject; that is, assessing whether or not renal function is improving or worsening in a subject who has suffered from an injury to renal function, reduced renal function, or ARF. In these aspects, the assay result(s), for example a measured concentration of CCL14 is correlated to the occurrence or nonoccurrence of a change in renal status. For example, the measured concentration of CCL14 is compared to a threshold value. When the measured concentration is above the threshold, a worsening of renal function is assigned to the subject; alternatively, when the measured concentration is below the threshold, an improvement of renal function is assigned to the subject.

A variety of methods may be used by the skilled artisan to arrive at a desired threshold value for use in these methods. For example, the threshold value is determined from a population of normal subjects by selecting a concentration representing the 75^{th}, 85^{th}, 90^{th}, 95^{th}, or 99^{th} percentile of CCL14 measured in such normal subjects. Alternatively, the threshold value is determined from a “diseased” population of subjects, e.g., those suffering from an injury or having a predisposition for an injury (e.g., progression to ARF or some other clinical outcome such as death, dialysis, renal transplantation, etc.), by selecting a concentration representing the 75^{th}, ⁸⁵th, 90^{th}, 95^{th}, or 99^{th} percentile of CCL14 measured in such subjects. In another alternative, the threshold value is determined from a prior measurement of CCL14 in the same subject; that is, a temporal change in the level of CCL14 in the subject is used to assign risk to the subject. Various methods of determining a threshold value are described, for example, in International publication numbers WO 2016/064877 and WO 2018/132702 and U.S. publication number 2018/0209990, which are incorporated by reference herein in their entirety.

### CCL14 Assays

In general, immunoassays involve contacting a sample containing or suspected of containing a biomarker of interest, such as CCL14, with at least one antibody that specifically binds to the biomarker. A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to bioMérieux VIDAS®, Beckman ACCESS®, Abbott AXSYM®, Roche ELECSYS®, Dade Behring STRATUS® systems are among the immunoassay analyzers that are capable of performing immunoassays. But any suitable immunoassay may be utilized, for example, enzyme-linked immunoassays (ELISA), radioimmunoassays (RIAs), enzyme-linked fluorescent assays (ELFA), competitive binding assays, and the like.

Antibodies or other polypeptides may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TENTAGELS™, AGROGELS®, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the latter case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that are indirectly detected by production of a detectable reaction product (e.g., enzymes such as horseradish peroxidase, alkaline phosphatase, etc.) or by a specific binding molecule which itself is detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In certain aspects, the present invention provides kits for the analysis of the described marker. The kit comprises reagents for the analysis of at least one test sample which comprise at least one antibody that specifically binds to the marker. The kit can also include devices and instructions for performing one or more of the diagnostic and/or prognostic correlations described herein. Preferred kits will comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies that bind a kidney injury marker. Most preferably each of the antibodies are monoclonal antibodies. The instructions for use of the kit and performing the correlations can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

The antibodies provided herein can be used in a lateral flow assay. The term “lateral flow” as used herein refers to flow of reagents in a longitudinal direction through a substantially flat porous material. Such porous material is “substantially flat” if the thickness of the material is no more than 10% of the length and width dimensions.

A lateral flow assay can be performed in a device. A lateral flow device can comprise different regions. The “sample application region” as used herein refers to a portion of an assay device into which a fluid sample of interest is introduced for purposes of determining a component thereof. The “downstream region” as used herein relative to a first region of a device refers to which receives fluid flow after that fluid has already reached the first region.

Representative lateral flow devices include those described in International publication numbers WO 2014/070935, WO 2014/134033; and U.S. publication numbers 2015/0293085, 2017/0234867, and 2016/0011188, which are incorporated by reference herein with respect to the lateral flow device design and function.

In certain aspects, the marker assay is performed using a single-use disposable test device. Such a test device may be a lateral flow device. Generally, these assay devices have an extended base layer on which a differentiation can be made between a sample addition region and an evaluation region. In typical use, the sample is applied to the sample addition region/sample application region, flows along a liquid transport path which runs parallel to the base layer, and then flows into the evaluation region. A capture reagent is present in the evaluation region, and the captured analyte can be detected by a variety of protocols to detect visible moieties associated with the captured analyte. For example, the assay may produce a visual signal, such as color change, fluorescence, luminescence, and the like, when indicating the presence or absence of an analyte in a sample, including, for example, a body fluid sample.

A sample addition region can be provided, for example, in the form of an open chamber in a housing; in the form of an absorbent pad; etc. The sample addition region can be a port of various configurations, that is, round, oblong, square and the like or the region can be a trough in the device.

A filter element can be placed in, on, or adjacent to the sample addition region to filter particulates from the sample, such as to remove or retard blood cells from blood so that plasma can further travel through the device. Filtrate can then move into a porous member fluidly connected to the filter. Suitable filters for removing or retarding cellular material present in blood are well known in the art. See, e.g., U.S. Pat. Nos. 4,477,575; 5,166,051; 6,391,265; and 7,125,493, each of which is hereby incorporated by reference in its entirety. Many suitable materials are known to skilled artisans, and can include glass fibers, synthetic resin fibers, membranes of various types including asymmetric membrane filters in which the pore size varies from about 65 to about 15 μm, and combinations of such materials. In addition, a filter element can comprise one or more chemical substances to facilitate separation of red blood cells from blood plasma. Examples of such chemical substances are thrombin, lectins, cationic polymers, antibodies against one or more red blood cell surface antigens and the like. Such chemical substance(s) which facilitate separation of red blood cells from plasma may be provided in the filter element by covalent means, nonspecific absorption, etc.

In certain aspects, a label zone is located downstream of the sample receiving zone, and contains a diffusively located labeled reagent that binds to the analyte of interest or that competes with the analyte of interest for binding to a binding species. Alternatively, the label zone can be eliminated if the labeled reagent is premixed with the sample prior to application to the sample receiving zone. A detection zone is disposed downstream of from the label zone, and contains an immobilized capture reagent that binds to the analyte of interest.

The optimum pore diameter for the membrane for use in the invention is about 10 to about 50 μm. The membranes typically are from about 1 mil to about 15 mils in thickness, typically in the range of from 5 or 10 mils, but may be up to 200 mils and thicker. The membrane may be backed by a generally water impervious layer, such as a Mylar® polyester film (DuPont Teijin Films). When employed, the backing is generally fastened to the membrane by an adhesive, such as 3M 444 double-sided adhesive tape. Typically, a water impervious backing is used for membranes of low thickness. A wide variety of polymers may be used provided that they do not bind nonspecifically to the assay components and do not interfere with flow of the sample. Illustrative polymers include polyethylene, polypropylene, polystyrene and the like. Alternatively, the membrane is self-supporting. Other non-bibulous membranes, such as polyvinyl chloride, polyvinyl acetate, copolymers of vinyl acetate and vinyl chloride, polyamide, polycarbonate, polystyrene, and the like, can also be used. In various aspects, the label zone material is pretreated with a solution that includes blocking and stabilizing agents. Blocking agents include bovine serum albumin (BSA), methylated BSA, casein, nonfat dry milk. The device can also comprise additional components, including for example buffering agents, HAMA inhibitors, detergents, salts (e.g., chloride and/or sulfate salts of calcium, magnesium, potassium, etc.), and proteinaceous components (e.g., serum albumin, gelatin, milk proteins, etc.). This list is not meant to be limiting.

The device may further comprise various control locations which are read to determine that the test device has been run properly. By way of example, a procedural control zone is provided separate from the assay detection zone to verify that the sample flow is as expected. The control zone is preferably a spatially distinct region at which a signal is generated that is indicative of the proper flow of reagents. The procedural control zone may contain the analyte of interest, or a fragment thereof, to which excess labeled antibody used in the analyte assay can bind. In operation, a labeled reagent binds to the control zone, even when the analyte of interest is absent from the test sample. The use of a control line is helpful in that appearance of a signal in the control line indicates the time at which the test result can be read, even for a negative result. Thus, when the expected signal appears in the control line, the presence or absence of a signal in the capture zone can be noted. The device may further comprise a negative control area. The purpose of this control area is to alert the user that the test device is not working properly. When working properly, no signal or mark should be visible in the negative control area.

The outer casing or housing of such an assay device may take various forms. Typically, it will include an elongate casing and may have a plurality of interfitting parts. In a particularly preferred aspect, the housing includes a top cover and a bottom support. The top cover contains an application aperture and an observation port. In a preferred aspect, the housing is made of moisture impervious solid material, for example, a plastic material. It is contemplated that a variety of commercially available plastics, including, but not limited to, vinyl, nylon, polyvinyl chloride, polypropylene, polystyrene, polyethylene, polycarbonates, polysulfanes, polyesters, urethanes, and epoxies maybe used to construct a housing. The housing may be prepared by conventional methodologies, such as standard molding technologies that are well known and used in the art. The housing may be produced by molding technologies which include, but are not limited to, injection molding, compression molding, transfer molding, blow molding, extrusion molding, foam molding, and thermoform molding. The aforementioned molding technologies are well known in the art and so are not discussed in detail herein. See for example, Processes And Materials Of Manufacture, Third Edition, R. A. Lindsberg (1983) Allyn and Baron pp. 393-431.

If necessary, the colorimetric, luminescent, or fluorescent intensity of the detectable label being employed may be then evaluated with an instrument that is appropriate to the label. By way of example, a fluorometer can be used to detect fluorescent labels; a reflectometer can be used to detect labels which absorb light, etc. The concentration of the analyte of interest in the samples may be determined by correlating the measured response to the amount of analyte in the sample fluid.

The antibodies of the present invention comprise special technical effects in their ability to perform in assays for human CCL14 in samples obtained from a subject, including, for example, body fluid samples. These antibodies perform particularly well in assay applications under conditions in which the antibodies bind CCL14 with high affinity and cooperatively bind and sandwich CCL14 with other CCL14 antibodies. The CCL14 antibodies of the present invention effectively bind to human CCL14 as it exists in a conformation and state found in patient biological fluid samples, as opposed to purified human CCL14. For example, different epitopes may present differently between proteins naturally occurring within complex body fluid samples and purified proteins. A minimum detectable concentration of human CCL14 within a sample can be one way of measuring performance of the antibodies of the present invention. In certain aspects, the antibodies of the invention perform unexpectedly well in sandwich assays, competitive assays, lateral flow devices and as components of kits.

### Assay Correlations

The terms “correlating” and “relating” as used herein in reference to the measurement of biomarkers in an assay refers to determining the presence, or more preferably the amount, of the biomarker in a sample based on the signal obtained from the assay. Often, this takes the form of comparing a signal generated from a detectable label on one species participating in the assay to a predetermined standard curve which can be used to convert the signal to a concentration or threshold amount of the biomarker.

The terms “correlating” and “relating” as used herein in reference to the use of biomarkers for diagnosis or prognosis refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy which is highly efficacious and has a low level of risk, few tests are needed because clinicians can accept substantial diagnostic uncertainty. On the other hand, in situations where treatment options are less effective and riskier, clinicians often need a higher degree of diagnostic certainty. Thus, cost/benefit analysis is involved in selecting a diagnostic threshold.

Suitable thresholds may be determined in a variety of ways. For example, one recommended diagnostic threshold for the diagnosis of acute myocardial infarction using cardiac troponin is the 97.5th percentile of the concentration seen in a normal population. Another method may be to look at serial samples from the same patient, where a prior “baseline” result is used to monitor for temporal changes in a biomarker level.

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1−specificity, the ROC graph is sometimes called the sensitivity vs (1−specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease is assigned with relatively high confidence, and above a second threshold the presence of disease is assigned with relatively high confidence. Between the two thresholds may be considered indeterminate. This is meant to be exemplary in nature only.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.8, even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

### Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs).

As used herein, the assignment of amino acids to each domain, framework region and CDR may be in accordance with one of the schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; Dubel, Ed. (2007) Handbook of Therapeutic Antibodies, 3^{rd} Ed., Wily-VCH Verlag GmbH and Co; or Lefranc et al., 2003 (IMGT numbering) Dev. Comp. Immunol. 27:55-57, unless otherwise noted. As is well known in the art variable region residue numbering is typically as set forth in Chothia, Kabat or Lefranc (IMGT). Amino acid residues which comprise CDRs as defined by Kabat, Chothia, MacCallum (also known as Contact) and Lefranc (also known as IMGT) are set out below in Table 1.

TABLE 1

| | | | | Lefranc |
|---|---|---|---|---|
| | Kabat | Chothia | MacCallum | (IMGT) |
| VH CDR1 | 31-35 | 26-32 | 30-35 | 27-38 |
| VH CDR2 | 50-65 | 52-56 | 47-58 | 56-65 |
| VH CDR3 | 95-102 | 95-102 | 93-101 | 105-117 |
| VL CDR1 | 24-34 | 24-34 | 30-36 | 27-38 |
| VL CDR2 | 50-56 | 50-56 | 46-55 | 56-65 |
| VL CDR3 | 89-97 | 89-97 | 89-96 | 105-117 |

Variable regions and CDRs in an antibody sequence can be identified according to general rules that have been developed in the art (as set out above, such as, for example, the Kabat nomenclature system) or by aligning the sequences against a database of known variable regions. Methods for identifying these regions are described in Lefranc et al., 2003, Dev. Comp. Immunol. 27:55-77; Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “IMGT” (ImMunoGeneTics) website at www.imgt.org; the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005).

Unless otherwise indicated, all CDRs set forth herein are derived according to the IMGT website as per Lefranc et al.

Preferred therapeutic antibodies are IgG antibodies. The term “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3. The known Ig domains in the IgG class of antibodies are VH, Cγ1, Cγ2, Cγ3, VL, and CL. IgG is the preferred class for therapeutic antibodies for several practical reasons. IgG antibodies are stable, easily purified, and able to be stored under conditions that are practical for pharmaceutical supply chains. In vivo they have a long biological half-life that is not just a function of their size but is also a result of their interaction with the so-called Fc receptor (or FcRn). This receptor seems to protect IgG from catabolism within cells and recycles it back to the plasma.

Antibodies are immunological proteins that bind a specific antigen. In most mammals, including humans and mice, antibodies are constructed from paired heavy and light polypeptide chains. The light and heavy chain variable regions show significant sequence diversity between antibodies, and are responsible for binding the target antigen. Each chain is made up of individual immunoglobulin (Ig) domains, and thus the generic term immunoglobulin is used for such proteins.

Antibodies for use in the claimed methods may be obtained from a variety of species. For example, the antibodies of the present invention may comprise immunoglobulin sequences which are rabbit, mouse, rat, guinea pig, chicken, goat, sheep, donkey, human, llama or camelid.

The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred aspects, Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

Antibodies of the invention may be further characterized by epitope mapping, so that antibodies and epitopes are selected that have the greatest clinical utility in the immunoassays described herein. The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. Preferably, an epitope is targeted which is present on the target molecule, but is partially or totally absent on non-target molecules.

In some aspects, the antibody scaffold can be a mixture of sequences from different species. Such an antibody may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8. Humanization or other methods of reducing the immunogenicity of nonhuman antibody variable regions may include resurfacing methods, as described for example in Roguska et al., 1994, Proc. Natl. Acad. Sci. USA 91:969-973. In one aspect, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759. Other humanization methods may involve the grafting of only parts of the CDRs, including but not limited to methods described in U.S. Ser. No. 09/810,502; Tan et al., 2002, J. Immunol. 169:1119-1125; De Pascalis et al., 2002, J. Immunol. 169:3076-3084.

In one aspect, the antibody is a fully human antibody. “Fully human antibody” or “complete human antibody” refers to a human antibody having the gene sequence of an antibody derived from a human chromosome. Fully human antibodies may be obtained, for example, using transgenic mice (Bruggemann et al., 1997, Curr Opin Biotechnol 8:455-458) or human antibody libraries coupled with selection methods (Griffiths et al., 1998, Curr Opin Biotechnol 9:102-108).

### Production of Antibodies

Monoclonal antibody preparations can be produced using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS, pp. 563-681 (Elsevier, N.Y., 1981) (both of which are incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Monoclonal antibodies derived from animals other than rats and mice offer unique advantages. Many protein targets relevant to signal transduction and disease are highly conserved between mice, rats and humans, and can therefore be recognized as self-antigens by a mouse or rat host, making them less immunogenic. This problem may be avoided when using rabbit as a host animal. See, e.g., Rossi et al., Am. J. Clin. Pathol., 124, 295-302, 2005.

Methods for producing and screening for specific antibodies using hybridoma technology are routine and well known in the art. In a non-limiting example, mice are immunized with an antigen of interest or a cell expressing such an antigen. Once an immune response is detected, e.g., antibodies specific for the antigen are detected in the mouse serum, the mouse spleen is harvested and splenocytes isolated. The splenocytes are then fused by well-known techniques to any suitable myeloma cells. Hybridomas are selected and cloned by limiting dilution. The hybridoma clones are then assayed by methods known in the art for cells that secrete antibodies capable of binding the antigen. Ascites fluid, which generally contains high levels of antibodies, can be generated by inoculating mice intraperitoneally with positive hybridoma clones.

Adjuvants that can be used in the methods of antibody generation include, but are not limited to, protein adjuvants; bacterial adjuvants, e.g., whole bacteria (BCG, Corynebacterium parvum, Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, iodoacetate and cholesteryl hemisuccinateor; naked DNA adjuvants. Other adjuvants that can be used in the methods of the invention include, Cholera toxin, paropox proteins, MF-59 (Chiron Corporation; See also Bieg et al. (1999) “GAD65 And Insulin B Chain Peptide (9-23) Are Not Primary Autoantigens In The Type 1 Diabetes Syndrome Of The BB Rat,” Autoimmunity, 31(1):15-24, which is incorporated herein by reference), MPL® (Corixa Corporation; See also Lodmell et al.

(2000) “DNA Vaccination Of Mice Against Rabies Virus: Effects Of The Route Of Vaccination And The Adjuvant Monophosphoryl Lipid A (MPL),” Vaccine, 18: 1059-1066; Johnson et al. (1999) “3-O-Desacyl Monophosphoryl Lipid A Derivatives: Synthesis And Immunostimulant Activities,” Journal of Medicinal Chemistry, 42: 4640-4649; Baldridge et al. (1999) “Monophosphoryl Lipid A (MPL) Formulations For The Next Generation Of Vaccines,” Methods, 19: 103-107, all of which are incorporated herein by reference), RC-529 adjuvant (Corixa Corporation; the lead compound from Corixa's aminoalkyl glucosaminide 4-phosphate (AGP) chemical library, see also www.corixa.com), and DETOX™ adjuvant (Corixa Corporation; DETOX™ adjuvant includes MPL® adjuvant (monophosphoryl lipid A) and mycobacterial cell wall skeleton; See also Eton et al. (1998) “Active Immunotherapy With Ultraviolet B-Irradiated Autologous Whole Melanoma Cells Plus DETOX In Patients With Metastatic Melanoma,” Clin. Cancer Res. 4(3):619-627; and Gupta et al. (1995) “Adjuvants For Human Vaccines—Current Status, Problems And Future Prospects,” Vaccine, 13(14): 1263-1276, both of which are incorporated herein by reference).

Numerous publications discuss the use of phage display technology to produce and screen libraries of polypeptides for binding to a selected analyte. See, e.g., Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods, a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) is present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg et al. (1995) “Human Antibodies From Transgenic Mice,” Int. Rev. Immunol. 13:65-93, which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

### Recombinant Expression of Antibodies

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In selected aspects the nucleic acid is a cDNA molecule.

Nucleic acids of the invention can be obtained using standard molecular biology techniques. For antibodies expressed by hybridomas, cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

The instant invention also provides vectors comprising such nucleic acids described above, which may be operably linked to a promoter and other transcriptional regulatory and processing control elements. The invention also provides host cells harboring those vectors and host-expression systems.

Once a nucleic acid sequence encoding an antibody of the invention has been obtained, the vector for the production of the antibody may be produced by recombinant DNA technology using techniques well known in the art. Methods which are well known to those skilled in the art can be used to construct expression vectors containing the antibody coding sequences and appropriate transcriptional and translational control signals. These methods include, for example, in vitro recombinant DNA techniques, synthetic techniques, and in vivo genetic recombination. (See, for example, the techniques described in Sambrook et al, 1990, MOLECULAR CLONING, A LABORATORY MANUAL, 2d Ed., Cold Spring Harbor Laboratory, Cold Spring Harbor, N.Y. and Ausubel et al. eds., 1998, CURRENT PROTOCOLS IN MOLECULAR BIOLOGY, John Wiley & Sons, NY).

An expression vector comprising the nucleotide sequence of an antibody can be transferred to a host cell by conventional techniques (e.g., electroporation, liposomal transfection, and calcium phosphate precipitation) and the transfected cells are then cultured by conventional techniques to produce the antibody of the invention. In specific aspects, the expression of the antibody is regulated by a constitutive, an inducible or a tissue, specific promoter.

The anti-CCL14 antibodies disclosed herein may also be produced recombinantly (e.g., in an E. coli/T7 expression system, a mammalian cell expression system or a lower eukaryote expression system). In this aspect, nucleic acids encoding the antibody immunoglobulin molecules of the invention (e.g., VH or VL) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an aspect of the invention, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside).

Thus, the present invention includes recombinant methods for making an anti-CCL14 antibody or antigen-binding fragment thereof of the present invention, or an immunoglobulin chain thereof, comprising introducing a polynucleotide encoding one or more immunoglobulin chains of the antibody or fragment (e.g., heavy and/or light immunoglobulin chain); culturing the host cell (e.g., CHO or Pichia or Pichia pastoris) under condition favorable to such expression and, optionally, isolating the antibody or fragment or chain from the host cell and/or medium in which the host cell is grown.

Anti-CCL14 antibodies can also be synthesized by any of the methods set forth in U.S. Pat. No. 6,331,415.

Eukaryotic and prokaryotic host cells, including mammalian cells as hosts for expression of the antibodies or fragments or immunoglobulin chains disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody or fragment or chain in the host cells or secretion of the into the culture medium in which the host cells are grown.

A variety of host-expression vector systems may be utilized to express the antibodies of the invention. Such host-expression systems represent vehicles by which the coding sequences of the antibodies may be produced and subsequently purified, but also represent cells which may, when transformed or transfected with the appropriate nucleotide coding sequences, express the antibodies of the invention in situ. These include, but are not limited to, microorganisms such as bacteria (e.g., E. coli and B. subtilis) transformed with recombinant bacteriophage DNA, plasmid DNA or cosmid DNA expression vectors containing immunoglobulin coding sequences; yeast (e.g., Saccharomyces pichia) transformed with recombinant yeast expression vectors containing immunoglobulin coding sequences; insect cell systems infected with recombinant virus expression vectors (e.g., baculovirus) containing the immunoglobulin coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing immunoglobulin coding sequences; or mammalian cell systems (e.g., COS, CHO, BHK, 293, 293T, 3T3 cells, lymphotic cells (see U.S. Pat. No. 5,807,715), Per C.6 cells (rat retinal cells developed by Crucell)) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody being expressed. For example, when a large quantity of such a protein is to be produced, for the generation of pharmaceutical compositions of an antibody, vectors which direct the expression of high levels of fusion protein products that are readily purified may be desirable. Such vectors include, but are not limited, to the E. coli expression vector pUR278 (Ruther et al. (1983) “Easy Identification Of cDNA Clones,” EMBO J. 2:1791-1794), in which the antibody coding sequence may be ligated individually into the vector in frame with the lac Z coding region so that a fusion protein is produced; pIN vectors (Inouye et al. (1985) “Up-Promoter Mutations In The Lpp Gene Of Escherichia coli,” Nucleic Acids Res. 13:3101-3110; Van Heeke et al. (1989) “Expression Of Human Asparagine Synthetase In Escherichia coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts. (See e.g., see Logan et al. (1984) “Adenovirus Tripartite Leader Sequence Enhances Translation Of mRNAs Late After Infection,” Proc. Natl. Acad. Sci. (U.S.A.) 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express an antibody of the invention may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells are allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibodies of the invention. Such engineered cell lines may be particularly useful in screening and evaluation of compounds that interact directly or indirectly with the antibodies of the invention.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11:223-232), hypoxanthine-guanine phosphoribosyltransferase (Szybalska et al. (1962) “Genetics Of Human Cess Line. IV. DNA-Mediated Heritable Transformation Of A Biochemical Trait,” Proc. Natl. Acad. Sci. (U.S.A.) 48:2026-2034), and adenine phosphoribosyltransferase (Lowy et al. (1980) “Isolation Of Transforming DNA: Cloning The Hamster Aprt Gene,” Cell 22:817-823) genes can be employed in tk−, hgprt− or aprt− cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. (U.S.A.) 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. (U.S.A.) 78:1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. (U.S.A.) 78:2072-2076); neo, which confers resistance to the aminoglycoside G-418 (Tachibana et al. (1991) “Altered Reactivity Of Immunoglobulin Produced By Human-Human Hybridoma Cells Transfected By pSV2-Neo Gene,” Cytotechnology 6(3):219-226; Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human gene therapy,” Ann. Rev. Biochem. 62:191-217). Methods commonly known in the art of recombinant DNA technology which can be used are described in Ausubel et al. (eds.), 1993, CURRENT PROTOCOLS IN MOLECULAR BIOLOGY, John Wiley & Sons, NY; Kriegler, 1990, GENE TRANSFER AND EXPRESSION, A LABORATORY MANUAL, Stockton Press, NY; and in Chapters 12 and 13, Dracopoli et al. (eds), 1994, CURRENT PROTOCOLS IN HUMAN GENETICS, John Wiley & Sons, NY; Colbere-Garapin et al. (1981) “A New Dominant Hybrid Selective Marker For Higher Eukaryotic Cells,” J. Mol. Biol. 150:1-14; and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156).

The expression levels of an antibody of the invention can be increased by vector amplification (for a review, see Bebbington and Hentschel, “The Use Of Vectors Based On Gene Amplification For The Expression Of Cloned Genes In Mammalian Cells,” in DNA CLONING, Vol. 3. (Academic Press, New York, 1987)). When a marker in the vector system expressing an antibody is amplifiable, increase in the level of inhibitor present in culture of host cell will increase the number of copies of the marker gene. Since the amplified region is associated with the nucleotide sequence of the antibody, production of the antibody will also increase (Crouse et al. (1983) “Expression And Amplification Of Engineered Mouse Dihydrofolate Reductase Minigenes,” Mol. Cell. Biol. 3:257-266).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot (1986) “Expression And Amplification Of Engineered Mouse Dihydrofolate Reductase Minigenes,” Nature 322:562-565; Kohler (1980) “Immunoglobulin Chain Loss In Hybridoma Lines,” Proc. Natl. Acad. Sci. (U.S.A.) 77:2197-2199). The coding sequences for the heavy and light chains may comprise cDNA or genomic DNA.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, comprise the instant invention, independent of the glycosylation pattern the antibodies may have. Similarly, in particular aspects, antibodies with a glycosylation pattern comprising only non-fucosylated N-glycans may be advantageous, because these antibodies have been shown to typically exhibit more potent efficacy than their fucosylated counterparts both in vitro and in vivo (See for example, Shinkawa et al., J. Biol. Chem. 278: 3466-3473 (2003); U.S. Pat. Nos. 6,946,292 and 7,214,775). These antibodies with non-fucosylated N-glycans are not likely to be immunogenic because their carbohydrate structures are a normal component of the population that exists in human serum IgG.

Once the antibody of the invention has been recombinantly expressed, it may be purified by any method known in the art for purification of an antibody, for example, by chromatography (e.g., ion exchange, affinity, particularly by affinity for the specific antigen after Protein A, and sizing column chromatography), centrifugation, differential solubility, or by any other standard technique for the purification of proteins.

### Antibody Conjugates

The anti-CCL14 antibodies and antigen-binding fragments thereof disclosed herein may also be conjugated to a chemical moiety. The chemical moiety may be, inter alia, a polymer, a radionuclide or a cytotoxic factor. In particular aspects, the chemical moiety is a polymer which increases the half-life of the antibody or fragment in the body of a subject. Suitable polymers include, but are not limited to, hydrophilic polymers which include but are not limited to polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antigen-binding fragments thereof disclosed herein may also be conjugated with labels such as ⁹⁹Tc, ⁹⁰Y, ³²P, 14^{C}, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Se, ¹⁰⁹Pd, ²³⁴Th, and ⁴⁰K, ¹⁵⁷Gd, ⁵⁵Mn, ⁵²Tr, and ⁵⁶Fe.

The antibodies and antigen-binding fragments disclosed herein may also be PEGylated, for example to increase its biological (e.g., serum) half-life. To PEGylate an antibody or fragment, the antibody or fragment, typically is reacted with a reactive form of polyethylene glycol (PEG), such as a reactive ester or aldehyde derivative of PEG, under conditions in which one or more PEG groups become attached to the antibody or antibody fragment. In particular aspects, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain aspects, the antibody or fragment to be PEGylated is an aglycosylated antibody or fragment. Methods for PEGylating proteins are known in the art and can be applied to the antibodies of the invention. See, e.g., EP 0 154 316 and EP 0 401 384.

The antibodies and antigen-binding fragments disclosed herein may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antigen-binding fragments thereof of the invention may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibodies and antigen-binding fragments thereof of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies and fragments are conventional and very well known in the art.

### Therapeutic Uses of Anti-CCL14 Antibodies

Further provided are methods for treating subjects, including human subjects, in need of treatment with the isolated antibodies or antigen-binding fragments thereof disclosed herein.

To prepare pharmaceutical or sterile compositions of the anti-CCL14 antibodies and antigen-binding fragments of the invention (e.g., antibodies 5H2/5K3, 8H3/8K3, 9H3/9K2, 14H1/14K1, 15H1/15K3 or 24H1/24K1 and humanized versions thereof), the antibody or antigen-binding fragment thereof is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the antibodies of the invention, administered alone or in combination with another therapeutic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further aspect, a further therapeutic agent that is administered to a subject in association with an anti-CCL14 antibody or antigen-binding fragment thereof of the invention (e.g., antibodies 5H2/5K3, 8H3/8K3, 9H3/9K2, 14H1/14K1, 15H1/15K3 or 24H1/24K1 or humanized versions thereof) in accordance with the Physicians' Desk Reference 2003 (Thomson Healthcare; 57th edition (Nov. 1, 2002)).

The mode of administration can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular aspects, the anti-CCL14 antibodies or antigen-binding fragments thereof of the invention (e.g., antibodies 5H2/5K3, 8H3/8K3, 9H3/9K2, 14H1/14K1, 15H1/15K3 or 24H1/24K1 or humanized versions thereof) can be administered by an invasive route such as by injection. In further aspects of the invention, an anti-CCL14 antibody or antigen-binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

The present invention provides a vessel (e.g., a plastic or glass vial, e.g., with a cap or a chromatography column, hollow bore needle or a syringe cylinder) comprising any of the antibodies or antigen-binding fragments of the invention (e.g., antibodies 5H2/5K3, 8H3/8K3, 9H3/9K2, 14H1/14K1, 15H1/15K3 or 24H1/24K1 or humanized versions thereof) or a pharmaceutical composition thereof. The present invention also provides an injection device comprising any of the antibodies or antigen-binding fragments of the invention (e.g., antibodies 5H2/5K3, 8H3/8K3, 9H3/9K2, 14H1/14K1, 15H1/15K3 or 24H1/24K1 or humanized versions thereof) or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., antibody or fragment or a pharmaceutical composition thereof), a needle for piecing skin and/or blood vessels for injection of the fluid; and a plunger for pushing the fluid out of the cylinder and through the needle bore. In an aspect of the invention, an injection device that comprises an antibody or antigen-binding fragment thereof of the present invention or a pharmaceutical composition thereof is an intravenous (IV) injection device. Such a device includes the antibody or fragment or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the patient through the cannula or trocar/needle. The antibody or fragment or a pharmaceutical composition thereof may, in an aspect of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an aspect of the invention, an injection device is an autoinjector; a jet injector or an external infusion pump. A jet injector uses a high-pressure narrow jet of liquid which penetrate the epidermis to introduce the antibody or fragment or a pharmaceutical composition thereof to a patient's body. External infusion pumps are medical devices that deliver the antibody or fragment or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. Such needleless devices comprising the pharmaceutical composition are also part of the present invention. The pharmaceutical compositions disclosed herein may also be administered by infusion. Examples of well-known implants and modules for administering the pharmaceutical compositions include those disclosed in: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art and those comprising the pharmaceutical compositions of the present invention are within the scope of the present invention.

Alternately, one may administer the anti-CCL14 antibody or antigen-binding fragment of the invention (e.g., antibodies 5H2/5K3, 8H3/8K3, 9H3/9K2, 14H1/14K1, 15H1/15K3 or 24H1/24K1 or humanized versions thereof) in a local rather than systemic manner, for example, via injection of the antibody or fragment directly into a tumor, e.g., a CCL14⁺ tumor. Furthermore, one may administer the antibody or fragment in a targeted drug delivery system, for example, in a liposome coated with a tissue-specific antibody, targeting, for example, a tumor e.g., a CCL14⁺ tumor, e.g., characterized by immunopathology. The liposomes will be targeted to and taken up selectively by the afflicted tissue. Such methods and liposomes are part of the present invention.

The administration regimen depends on several factors, including the serum or tissue turnover rate of the therapeutic antibody or antigen-binding fragment, the level of symptoms, the immunogenicity of the therapeutic antibody, and the accessibility of the target cells in the biological matrix. Preferably, the administration regimen delivers sufficient therapeutic antibody or fragment to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies or fragments is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, humanized and fully human antibodies may be desirable.

Antibodies or antigen-binding fragments thereof disclosed herein (e.g., antibodies 5H2/5K3, 8H3/8K3, 9H3/9K2, 14H1/14K1, 15H1/15K3 or 24H1/24K1 or humanized versions thereof) may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:151-144). Doses may also be provided to achieve a pre-determined target concentration of anti-CCL14 antibody in the subject's serum, such as 0.1, 0.3, 1, 3, 10, 30, 100, 300 μg/ml or more. In other aspects, an anti-CCL14 antibody of the present invention is administered, e.g., subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, the term “effective amount” refer to an amount of an anti-CCL14 or antigen-binding fragment thereof of the invention (e.g., antibodies 5H2/5K3, 8H3/8K3, 9H3/9K2, 14H1/14K1, 15H1/15K3 or 24H1/24K1 or humanized versions thereof) that, when administered alone or in combination with an additional therapeutic agent to a cell, tissue, or subject, is effective to cause a measurable improvement in one or more symptoms of disease, for example cancer or the progression of cancer. An effective dose further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Experimental and Diagnostic Uses

The anti-CCL14 antibodies and antigen-binding fragments thereof disclosed herein may be used as affinity purification agents. In this process, the anti-CCL14 antibodies and antigen-binding fragments thereof are immobilized on a solid phase such a Sephadex, glass or agarose resin or filter paper, using methods well known in the art. The immobilized antibody or fragment is contacted with a sample containing the CCL14 protein (or a fragment thereof) to be purified, and thereafter the support is washed with a suitable solvent that will remove substantially all the material in the sample except the CCL14 protein, which is bound to the immobilized antibody or fragment. Finally, the support is washed with a solvent which elutes the bound CCL14 (e.g., protein A). Such immobilized antibodies and fragments form part of the present invention.

Further provided are antigens for generating secondary antibodies which are useful for example for performing Western blots and other immunoassays discussed herein.

Anti-CCL14 antibodies (e.g., humanized antibodies) and antigen-binding fragments thereof may also be useful in diagnostic assays for CCL14 protein, e.g., detecting its expression in specific cells, tissues, or serum, e.g., tumor cells such as melanoma cells. Such diagnostic methods may be useful in various disease diagnoses.

The present invention includes methods of detecting CCL14 in a sample obtained from a subject having acute kidney injury using any of the anti-CCL14 antibodies provided herein. The methods comprise contacting a sample with one or more of the anti-CCL14 antibodies of the present invention and generating an assay result. In some aspects, the assay result is a measured concentration of CCL14. The CCL14 can be detected by any of the various assays provided in the present invention. In some aspects, the assay is an immunoassay. In further aspects, the assay is a sandwich assay or a competitive assay.

The present invention includes ELISA assays (enzyme-linked immunosorbent assay) incorporating the use of an anti-CCL14 antibody or antigen-binding fragment thereof disclosed herein (e.g., antibodies 5H2/5K3, 8H3/8K3, 9H3/9K2, 14H1/14K1, 15H1/15K3 or 24H1/24K1 or a humanized version thereof).

For example, such a method comprises the following steps:
(a) coat a substrate (e.g., surface of a microtiter plate well, e.g., a plastic plate) with anti-CCL14 antibody or antigen-binding fragment thereof;
(b) apply a sample to be tested for the presence of CCL14 to the substrate;
(c) wash the plate, so that unbound material in the sample is removed;
(d) apply detectably labeled antibodies (e.g., enzyme-linked antibodies) which are also specific to the CCL14 antigen;
(e) wash the substrate, so that the unbound, labeled antibodies are removed;
(f) if the labeled antibodies are enzyme linked, apply a chemical which is converted by the enzyme into a fluorescent signal; and
(g) detect the presence of the labeled antibody.
   Detection of the label associated with the substrate indicates the presence of the CCL14 protein.

In a further aspect, the labeled antibody or antigen-binding fragment thereof is labeled with peroxidase which react with ABTS (e.g., 2,2′-azino-bis(3-ethylbenzthiazoline-6-sulphonic acid)) or 3,3′,5,5′-Tetramethylbenzidine to produce a color change which is detectable. Alternatively, the labeled antibody or fragment is labeled with a detectable radioisotope (e.g., ³H) which can be detected by scintillation counter in the presence of a scintillant.

In another aspect, the CCL14 antibodies of the invention may be used in a competitive assay. A “competitive assay” is an assay in which the CCL14 in a sample competes for binding to an antibody with added CCL14. In such assays, the added CCL14 can be immobilized or labeled with a detectable label. Competitive assay formats are known in the art. Generally, competitive assays include various art-recognized techniques, such as, for example, immunoassays, such as radioimmunoassays and enzyme linked immunosorbent assay (ELISA). Such immunoassays are routine and well known in the art. See, for example, Cox, K. L, et al., “Immunoassay Methods” 2012, Assay Guidance Manual; PMID: 22553884.

By way of example, in a competitive assay format one or more of the anti-CCL14 antibodies of the invention is conjugated to a detectable label. CCL14 in the sample competes for binding to the antibody with CCL14 that is immobilized, and the amount of signal obtained will be inversely proportional to the amount of CCL14 in the sample. Alternatively, CCL14 is conjugated to a detectable label. CCL14 in the sample competes with the detectably labeled CCL14 for binding to an antibody that is immobilized. The unbound CCL14 is removed by washing with an appropriate buffer and the amount of signal obtained will be inversely proportional to the amount of CCL14 in the sample.

In one aspect, a representative competitive assay method for detecting CCL14 in a body fluid sample comprises the steps of: (a) contacting a body fluid sample obtained from a subject with any of the anti-CCL14 antibodies disclosed herein (e.g., any of antibodies 5H2/5K3, 8H3/8K3, 9H3/9K2, 14H1/14K1, 15H1/15K3 or 24H1/24K1), and with detectably labeled CCL14 protein, and wherein the CCL14 in the body fluid sample competes with the detectably labeled CCL14 for binding to the anti-CCL14 antibody; and (b) detecting the label so as to detect the CCL14 in the body fluid sample.

In another aspect, a representative competitive assay method for detecting CCL14 in a body fluid sample comprises the steps of: (a) contacting a body fluid sample obtained from a subject with any of the anti-CCL14 antibodies disclosed herein (e.g., any of antibodies 5H2/5K3, 8H3/8K3, 9H3/9K2, 14H1/14K1, 15H1/15K3 or 24H1/24K1), and with immobilized CCL14 protein, wherein the CCL14 in the body fluid sample competes with the immobilized CCL14 for binding to the anti-CCL14 antibody, and wherein the anti-CCL14 antibody is detectably labeled; and (b) detecting the label so as to detect the CCL14 in the body fluid sample.

An anti-CCL14 antibody or antigen-binding fragment thereof of the invention may be used in a Western blot or immune-protein blot procedure. Such a procedure forms part of the present invention and includes e.g., optionally transferring proteins from a sample to be tested for the presence of CCL14 (e.g., from a PAGE or SDS-PAGE electrophoretic separation of the proteins in the sample) onto a membrane or other solid substrate using a method known in the art (e.g., semi-dry blotting or tank blotting); contacting the membrane or other solid substrate to be tested for the presence of bound CCL14 or a fragment thereof with an anti-CCL14 antibody or antigen-binding fragment thereof of the invention; washing the membrane one or more times to remove unbound anti-CCL14 antibody or fragment and other unbound substances; and detecting the bound anti-CCL14 antibody or fragment.

Such a membrane may take the form of a nitrocellulose or vinyl-based (e.g., polyvinylidene fluoride (PVDF)) membrane to which the proteins to be tested for the presence of CCL14 in a non-denaturing PAGE (polyacrylamide gel electrophoresis) gel or SDS-PAGE (sodium dodecyl sulfate polyacrylamide gel electrophoresis) gel have been transferred (e.g., following electrophoretic separation in the gel). Before contacting the membrane with the anti-CCL14 antibody or fragment, the membrane is optionally blocked, e.g., with non-fat dry milk or the like so as to bind non-specific protein binding sites on the membrane.

Detection of the bound antibody or fragment indicates that the CCL14 protein is present on the membrane or substrate and in the sample. Detection of the bound antibody or fragment may be by binding the antibody or fragment with a secondary antibody (an anti-immunoglobulin antibody) which is detectably labeled and, then, detecting the presence of the secondary antibody.

The anti-CCL14 antibodies and antigen-binding fragments thereof disclosed herein may also be used for immunohistochemistry. Such a method forms part of the present invention and comprises, e.g., contacting a cell (e.g., a tumor cell such as a melanoma cell) or a tissue sample to be tested for the presence of CCL14 protein with an anti-CCL14 antibody or antigen-binding fragment thereof of the invention; and detecting the antibody or fragment on or in the cell or tissue sample.

A representative immunohistochemistry method for detecting CCL14 in a tissue sample may comprise the steps of: (a) contacting a tissue sample obtained from a subject with any of the anti-CCL14 antibodies provided herein (e.g., any of antibodies 5H2/5K3, 8H3/8K3, 9H3/9K2, 14H1/14K1, 15H1/15K3 or 24H1/24K1); and (b) detecting the presence of the CCL14 antibody in the tissue sample.

If the antibody or fragment itself is detectably labeled, it can be detected directly. Alternatively, the antibody or fragment is bound by a detectably labeled secondary antibody which is detected.

In such immunohistochemistry methods, the tissue sample may be chemically fixed (including but not limited to: formaldehyde, gluteraldehyde, osmium tetroxide, potassium dichromate, acetic acid, alcohols, zinc salts, mercuric chloride, chromium tetroxide and picric acid) and embedded (including but not limited to: glycol methacrylate, paraffin and resins) or preserved via freezing.

Certain anti-CCL14 antibodies and antigen-binding fragments thereof disclosed herein may also be used for in vivo tumor imaging. Such a method may include injection of a radiolabeled anti-CCL14 antibody or antigen-binding fragment thereof into the body of a patient to be tested for the presence of a tumor associated with CCL14 expression (e.g., which expresses CCL14, for example, on the tumor cell surface) followed by nuclear imaging of the body of the patient to detect the presence of the labeled antibody or fragment e.g., at loci comprising a high concentration of the antibody or fragment which are bound to the tumor. The detection of the loci indicates the presence of the CCL14⁺ tumor and tumor cells.

Imaging techniques include SPECT imaging (single photon emission computed tomography) or PET imaging (positron emission tomography). Labels include e.g., iodine-123 (¹²³I) and technetium-99m (^{99m}Tc), e.g., in conjunction with SPECT imaging or ¹¹C, ¹³N, ¹⁵O or ¹⁸F, e.g., in conjunction with PET imaging or Indium-111 (See e.g., Gordon et al., (2005) International Rev. Neurobiol. 67:385-440).

### Pharmaceutical Compositions and Administration

To prepare pharmaceutical or sterile compositions of the anti-CCL14 antibodies and antigen-binding fragments of the invention, the antibody or antigen-binding fragment thereof is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the antibodies of the invention, administered alone or in combination with another therapeutic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further aspect, a further therapeutic agent that is administered to a subject in association with an anti-CCL14 antibody or antigen-binding fragment thereof of the invention in accordance with the Physicians' Desk Reference 2003 (Thomson Healthcare; 57th edition (Nov. 1, 2002)).

The mode of administration can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, intratumoral, or intra-arterial.

In particular aspects, the anti-CCL14 antibodies or antigen-binding fragments thereof of the invention can be administered by an invasive route such as by injection. In further aspects of the invention, an anti-CCL14 antibody or antigen-binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

The present invention provides a vessel (e.g., a plastic or glass vial, e.g., with a cap or a chromatography column, hollow bore needle or a syringe cylinder) comprising any of the antibodies or antigen-binding fragments of the invention or a pharmaceutical composition thereof. The present invention also provides an injection device comprising any of the antibodies or antigen-binding fragments of the invention or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., antibody or fragment or a pharmaceutical composition thereof), a needle for piecing skin and/or blood vessels for injection of the fluid; and a plunger for pushing the fluid out of the cylinder and through the needle bore. In an aspect of the invention, an injection device that comprises an antibody or antigen-binding fragment thereof of the present invention or a pharmaceutical composition thereof is an intravenous (IV) injection device. Such a device includes the antibody or fragment or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the patient through the cannula or trocar/needle. The antibody or fragment or a pharmaceutical composition thereof may, in an aspect of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an aspect of the invention, an injection device is an autoinjector; a jet injector or an external infusion pump. A jet injector uses a high-pressure narrow jet of liquid which penetrate the epidermis to introduce the antibody or fragment or a pharmaceutical composition thereof to a patient's body. External infusion pumps are medical devices that deliver the antibody or fragment or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. Such needleless devices comprising the pharmaceutical composition are also part of the present invention. The pharmaceutical compositions disclosed herein may also be administered by infusion. Examples of well-known implants and modules for administering the pharmaceutical compositions include those disclosed in: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art and those comprising the pharmaceutical compositions of the present invention are within the scope of the present invention.

Alternately, one may administer the anti-CCL14 antibody or antigen-binding fragment of the invention in a local rather than systemic manner, for example, via injection of the antibody or fragment directly into a tumor, e.g., a CCL14⁺ tumor. Furthermore, one may administer the antibody or fragment in a targeted drug delivery system, for example, in a liposome coated with a tissue-specific antibody, targeting, for example, a tumor e.g., a CCL14⁺ tumor, e.g., characterized by immunopathology. The liposomes will be targeted to and taken up selectively by the afflicted tissue. Such methods and liposomes are part of the present invention.

The administration regimen depends on several factors, including the serum or tissue turnover rate of the therapeutic antibody or antigen-binding fragment, the level of symptoms, the immunogenicity of the therapeutic antibody, and the accessibility of the target cells in the biological matrix. Preferably, the administration regimen delivers sufficient therapeutic antibody or fragment to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies or fragments is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, humanized and fully human antibodies are may be desirable.

Antibodies or antigen-binding fragments thereof disclosed herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:151-144). Doses may also be provided to achieve a pre-determined target concentration of anti-CCL14 antibody in the subject's serum, such as 0.1, 0.3, 1, 3, 10, 30, 100, 300 μg/ml or more. In other aspects, An anti-CCL14 antibody of the present invention is administered, e.g., subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, the term “effective amount” refer to an amount of an anti-CCL14 or antigen-binding fragment thereof of the invention that, when administered alone or in combination with an additional therapeutic agent to a cell, tissue, or subject, is effective to cause a measurable improvement in one or more symptoms of disease, for example cancer or the progression of cancer. An effective dose further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Kits

Further provided are kits comprising one or more components that include, but are not limited to, an anti-CCL14 antibody or antigen-binding fragment, as discussed herein in association with one or more additional components including, but not limited to a pharmaceutically acceptable carrier and/or a therapeutic agent, as discussed herein. The antibody or fragment and/or the therapeutic agent can be formulated as a pure composition or in combination with a pharmaceutically acceptable carrier, in a pharmaceutical composition.

In one aspect, the kit includes an anti-CCL14 antibody or antigen-binding fragment thereof of the invention or a pharmaceutical composition thereof in one container (e.g., in a sterile glass or plastic vial) and a pharmaceutical composition thereof and/or a therapeutic agent in another container (e.g., in a sterile glass or plastic vial).

In another aspect, the kit comprises a combination of the invention, including an anti-CCL14 antibody or antigen-binding fragment thereof of the invention along with a pharmaceutically acceptable carrier, optionally in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

As a matter of convenience, an anti-CCL14 antibody or antigen-binding fragment thereof of the invention can be provided in a kit, i.e., a packaged combination of reagents in predetermined amounts with instructions for performing the diagnostic or detection assay. Where the antibody or fragment is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

Also provided are diagnostic or detection reagents and kits comprising one or more such reagents for use in a variety of detection assays, including for example, immunoassays such as ELISA (sandwich-type or competitive format). The kit's components may be pre-attached to a solid support, or may be applied to the surface of a solid support when the kit is used. In some aspects of the invention, the signal generating means may come pre-associated with an antibody or fragment of the invention or may require combination with one or more components, e.g., buffers, antibody-enzyme conjugates, enzyme substrates, or the like, prior to use. Kits may also include additional reagents, e.g., blocking reagents for reducing nonspecific binding to the solid phase surface, washing reagents, enzyme substrates, and the like. The solid phase surface may be in the form of a tube, a bead, a microtiter plate, a microsphere, or other materials suitable for immobilizing proteins, peptides, or polypeptides. In particular aspects, an enzyme that catalyzes the formation of a chemiluminescent or chromogenic product or the reduction of a chemiluminescent or chromogenic substrate is a component of the signal generating means. Such enzymes are well known in the art. Kits may comprise any of the capture agents and detection reagents described herein. Optionally the kit may also comprise instructions for carrying out the methods of the invention.

Also provided is a kit comprising an anti-CCL14 antibody (e.g., humanized antibody) or antigen-binding fragment thereof packaged in a container, such as a vial or bottle, and further comprising a label attached to or packaged with the container, the label describing the contents of the container and providing indications and/or instructions regarding use of the contents of the container to treat one or more disease states as described herein.

In one aspect, the kit is for treating cancer and comprises an anti-CCL14 antibody (e.g., humanized antibody) or antigen-binding fragment thereof and a further therapeutic agent or a vaccine. The kit may optionally further include a syringe for parenteral, e.g., intravenous, administration. In another aspect, the kit comprises an anti-CCL14 antibody (e.g., humanized antibody) or antigen-binding fragment thereof and a label attached to or packaged with the container describing use of the antibody or fragment with the vaccine or further therapeutic agent. In yet another aspect, the kit comprises the vaccine or further therapeutic agent and a label attached to or packaged with the container describing use of the vaccine or further therapeutic agent with the anti-CCL14 antibody or fragment. In certain aspects, an anti-CCL14 antibody and vaccine or further therapeutic agent are in separate vials or are combined together in the same pharmaceutical composition.

As discussed above in the combination therapy section, concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The therapeutic and detection kits disclosed herein may also be prepared that comprise at least one of the antibody, peptide, antigen-binding fragment, or polynucleotide disclosed herein and instructions for using the composition as a detection reagent or therapeutic agent. Containers for use in such kits may typically comprise at least one vial, test tube, flask, bottle, syringe or other suitable container, into which one or more of the detection and/or therapeutic composition(s) may be placed, and preferably suitably aliquoted. Where a second therapeutic agent is also provided, the kit may also contain a second distinct container into which this second detection and/or therapeutic composition may be placed. Alternatively, a plurality of compounds may be prepared in a single pharmaceutical composition, and may be packaged in a single container means, such as a vial, flask, syringe, bottle, or other suitable single container. The kits disclosed herein will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the detection or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

A device or apparatus for carrying out the detection or monitoring methods described herein is also provided. Such an apparatus may include a chamber or tube into which sample can be input, a fluid handling system optionally including valves or pumps to direct flow of the sample through the device, optionally filters to separate plasma or serum from blood, mixing chambers for the addition of capture agents or detection reagents, and optionally a detection device for detecting the amount of detectable label bound to the capture agent immunocomplex. The flow of sample may be passive (e.g., by capillary, hydrostatic, or other forces that do not require further manipulation of the device once sample is applied) or active (e.g., by application of force generated via mechanical pumps, electroosmotic pumps, centrifugal force, or increased air pressure), or by a combination of active and passive forces.

In further aspects, also provided is a processor, a computer readable memory, and a routine stored on the computer readable memory and adapted to be executed on the processor to perform any of the methods described herein. Examples of suitable computing systems, environments, and/or configurations include personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, or any other systems known in the art.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992)J Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Single chain antibodies and diabodies are described (see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al. (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914).

Multispecific antibodies are also provided (see, e.g., Azzoni et al. (1998) J Immunol. 161:3493; Kita et al. (1999) J. Immunol. 162:6901; Merchant et al. (2000) J. Biol. Chem. 74:9115; Pandey et al. (2000) J. Biol. Chem. 275:38633; Zheng et al. (2001) J. Biol. Chem. 276:12999; Propst et al. (2000) J. Immunol. 165:2214; Long (1999) Ann. Rev. Immunol. 17:875); Labrijin et al., Proc. Natl. Acad. Sci. USA 110: 5145-50, 2013; de Jong et al., PLOS Biol 14(1): e1002344, 2016 (doi:10.1371/journal.pbio.1002344). Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Sequences

Table 2 is a summary of sequences referred to in the present invention.

TABLE 2

| | SEQ ID | |
|---|---|---|
| Description | NO: | SEQUENCE |
| 5H2 heavy chain | 1 | METGLRWLLLVAVLKGVQCQSVKESEGGLFKPT |
| variable region | | DTLTLTCTVSGFSLYSGAINWVRQAPGEGLQYIG |
| sequence (amino acid | | WISDVGSAYYASWAKSRSTITRNTNENTVTLKMT |
| sequence) | | SLTAADTATYFCARGDGSSGNYWVTDIWGQGTL |
| | | VTVSSGQPK |
| 5K3 light chain | 2 | MDTRAPTQLLGLLLLWLPGARCAVVLTQTASPVS |
| variable region | | APVGGTVTINCQASESISSRLAWYQQKPGQPPKLL |
| sequence (amino acid | | IYGASTLASGVPSRFKGSGSGTEFTLTISDLECADA |
| sequence) | | ATYSCQEYLSDNTFGGGTEVVVKGDPV |
| 8H3 heavy chain | 3 | METGLRWLLLVAVLKGVQCQSVKESEGGLFKPT |
| variable region | | DTLTLTCTVSGFSLYSGAINWVRQAPGEGLQYIG |
| sequence (amino acid | | WISDVGSAYYASWAKSRSTITRNTNENTVTLKMT |
| sequence) | | SLTAADTATYFCARGDGSSGNYWVTDIWGQGTL |
| | | VTVSSGQPK |
| 8K3 light chain | 4 | MDTRAPTQLLGLLLLWLPGARCAVVLTQTASPVS |
| variable region | | APVGGTVTINCQASESISSRLAWYQQKPGQPPKLL |
| sequence (amino acid | | IYGASTLASGVPSRFKGSGSGTEFTLTISDLECADA |
| sequence) | | ATYSCQEYLSDNTFGGGTEVVVKGDPV |
| 9H3 heavy chain | 5 | METGLRWLLLVAVLKGVQCQSVKESEGGLFKPT |
| variable region | | DTLTLTCTVSGFSLNSYGVNWVRQAPGNGLEYIG |
| sequence (amino acid | | TVGSSGSAYYASWAKSRSTITRNTNLNTVTLKMT |
| sequence) | | SLTAADTATYFCARGLIATMSIWGQGTLVTVSSG |
| | | QPK |
| 9K2 light chain | 6 | MDTRAPTQLLGLLLLWLPGATFAQVLTQTASSVS |
| variable region | | AAVGGTVTISCQSSQSVHSNNYLAWYQQKPGQPP |
| sequence (amino acid | | KQLIYLASTLASGVPSRFKGSGSGTQFTLTISDLEC |
| sequence) | | DDAATYYCAGGYSGMIFTFGGGTEVVVKGDPV |
| 14H1 heavy chain | 7 | METGLRWLLLVAVLKGVQCQSLEESGGDLVKPG |
| variable region | | AFLTLTCTASGFSFSGSDYMWWVRQAPGKGLEW |
| sequence (amino acid | | IACIYGGYSGSTYYASWAKGRFTISKTSSTTVTLQ |
| sequence) | | MTRLTAADTAIYFCARDGGVTHFSHFDLWGQGT |
| | | LVTVSSGQPK |
| 14K1 light chain | 8 | MDTRAPTQLLGLLLLWLPGARCDVVMTQTPASV |
| variable region | | SEPVGGTVTIKCQASEDIYRLLAWYQQKPGQPPK |
| sequence (amino acid | | LLIYGASTLASGVPSRFSGSGSGTEYTLTINDLECA |
| sequence) | | DAATYYCQYITYGSDVLTAFGGGTEVVVKGDPV |
| 15H1 heavy chain | 9 | METGLRWLLLVAVLKGVQCQSLEESGGDLVKPG |
| variable region | | ASLTLTCTASGFSFSRSYYVCWVRQAPGKGLEWI |
| sequence (amino acid | | VCIYGGSSDTTYYASWAKGRFTISKTSSTTVTLQL |
| sequence) | | NSLTAADTATYFCARRDVSGGYDYGMDLWGQG |
| | | TLVTVSSGQPK |
| 15K3 light chain | 10 | MDTRAPTQLLGLLLLWLPGARCAYDMTQTPASV |
| variable region | | EVAVGGTVTIKCQASEDIESYLAWYQQKPGQPPK |
| sequence (amino acid | | LLIYDASDLASGVPSRFKGSGSGTEYTLTISDLECD |
| sequence) | | DAATYYCRQGYSSSNVDNVFGGGTEVVVKGDPV |
| 24H1 heavy chain | 11 | METGLRWLLLVAVLKGVQCQSLEESGGDLVKPG |
| variable region | | ASLTLTCIGSGFDFSSNAIWWVRQAPGKGLEWIA |
| sequence (amino acid | | CLYGGTSGSTEYATWAKGRFTISKTSSTTVTLQM |
| sequence) | | TSLTDADTATYYCAGGVVTWSYPRQLYLWGQGT |
| | | LVTVSSGQPK |
| 24K1 light chain | 12 | MDTRAPTQLLGLLLLWLPGARCADVVMTQTPAS |
| variable region | | VSAVVGGTVTIKCQASQSISSWLSWYQQKLGQPP |
| sequence (amino acid | | KLLIYSASTLASGVPSRFKGSGSGADYTLTISDLEC |
| sequence) | | ADAATYYCQSNTAVHTYNFGGGTEVVVKGDPV |
| 5H2/5K3 epitope 1 | 13 | SRGPYHPSECCFTYT |
| sequence | | |
| 5H2/5K3 epitope 2 | 14 | YETNSQCSKPGIVFI |
| sequence | | |
| 8H3/8K3 epitope | 15 | YYETNSQCSKPGIVFI |
| sequence | | |
| 9H3/9K2 epitope | 16 | SDKWVQDYIKDMKE |
| sequence | | |
| 14H1/14K1 epitope 1 | 17 | CCFTYTTYKIPRQR |
| sequence | | |
| 14H1/14K1 epitope 2 | 18 | NSQCSKPGIVFIT |
| sequence | | |
| 15H1/15K3 and | 19 | TYKIPRQRIMDYYE |
| 24H1/24K1 epitope | | |
| sequence | | |
| Human CCL14 | 20 | MKISVAAIPFFLLITIALGTKTESSSRGPYHPSECCF |
| precursor amino acid | | TYTTYKIPRQRIMDYYETNSQCSKPGIVFITKRGHS |
| sequence | | VCTNPSDKWVQDYIKDMKEN |
| HCC-3 Domain of | 21 | QTGGKPKVVKIQLKLVG |
| CCL14 | | |

### EXAMPLES

### Example 1: Monoclonal Antibody Development in Rabbits

Female New Zealand Rabbits were immunized by subcutaneous injections (SQ) with antigen/adjuvant emulsions. Primary immunization was done with Complete Freund's Adjuvant and Incomplete Freund's Adjuvant was used for all subsequent boosts. Rabbits were injected SQ every three weeks at 250 μg CCL14 antigen per rabbit (alternating two sites, hips and scapulas). A test bleed was taken from the marginal ear vein seven days after the second boost. This test bleed (immune sera) was tested by indirect ELISA assay to determine if immune response of the rabbit was adequate for monoclonal antibody development. Responding rabbits were given a final SQ boost and four days later was euthanized via exsanguination. The whole blood was collected via cardiac puncture. B cells producing antibody of interest were identified by indirect ELISA on target antigen and immunoglobulin genes were isolated. Heavy and light chains were cloned into separate mammalian expression vectors, transfected into HEK cells (transient transfection), and tissue culture supernatant containing rabbit monoclonal antibodies were harvested. Heavy and light chain sequences were obtained by DNA sequencing.

### Example 2: Epitope Mapping of Monoclonal Antibodies to CCL14

The epitopes of various monoclonal antibodies of the invention were mapped using linear, conformational, and discontinuous mapping methods.

The concept of mapping linear epitopes using libraries of overlapping synthetic peptides was pioneered by Geysen and Meloen (PNAS 81: 3998-4002, 1984). Linear peptides were synthesized directly on a solid support covered with a proprietary hydrogel formulation.

To generate a first peptide library, the amino acid sequence of the CCL14 protein was first split into overlapping 15 residue fragments in silico with an offset of one residue, which were then synthesized on a solid support.

In a second library of peptides derived from the first library, each residue in a fragment at positions 10 and 11 were replaced by alanine (unless the native residue was alanine, in which case it is replaced by glycine).

In a third library of peptides derived from the first library, each cysteine was replaced by a Cys-acetamidomethyl residue.

For a fourth library of peptides, the amino acid sequence of the CCL14 protein was first split into overlapping 25 residue fragments in silico with an offset of one residue, which were then synthesized on a solid support.

For a fifth library, constrained peptides of length 17 were generated. On positions 2-16 are 15-mer peptides derived from the target sequence of CCL14 with an offset of one residue. Cys residues were inserted on positions 1 and 17 and joined by mP2 CLIPS in order to create a loop mimic. Native Cys are replaced by a Cys-acetamidomethyl residue.

For a sixth library, constrained peptides of length 21 were generated. On positions 2-16 are 19-mer peptides derived from the target sequence of CCL14 with an offset of one residue. Cys residues were inserted on positions 1 and 21 and joined by mP2 CLIPS in order to create a loop mimic. Native Cys are replaced by a Cys-acetamidomethyl residue.

For a seventh library, constrained peptides of length 27 were generated. On positions 2-26 are 25-mer peptides derived from the target sequence of CCL14 with an offset of one residue. Cys residues were inserted on positions 1 and 27 and joined by mP2 CLIPS in order to create a loop mimic. Native Cys are replaced by a Cys-acetamidomethyl residue. For an eighth library, (3-turn epitope mimics of length 22 were generated. On positions 2-21 are 20-mer peptides derived from the target sequence of CCL14 with an offset of one residue. Residues on positions 11 and 12 are replaced by a “PG” motif in order to induce the (3-turn formation. Cys residues were inserted on positions 1 and 22 and joined by mP2 CLIPS in order to stabilize the mimic. Native Cys are replaced by a Cys-acetamidomethyl residue.

For a ninth library, α-helical epitope mimics of length 22 were generated. Cys residues were inserted on positions 1 and 5 in order to nucleate an α-helical turn using mP2 CLIPS. Cys residues were inserted on positions 1 and 22 and joined by mP2 CLIPS in order to stabilize the mimic. Native Cys are replaced by a Cys-acetamidomethyl residue.

For a tenth library, peptides of length 25 derived from the CCL14 were generated. Each 25-mer peptide contains a pair of cysteine residues that are indicated to form a disulfide bridge as per UniProt information on post-translational modification of CCL14. Cys residues not participating in the disulfide bridge formation are replaced by a Cys-acetamidomethyl residue.

For an eleventh library, peptides of length 27 derived from the CCL14 were generated. Each 27-mer is composed of two 11-mer peptides joined via “GGSGG” linker. Two combined 11-mers contain a pair of cysteine residues that are indicated to form a disulfide bridge as per UniProt information on post-translational modification of CCL14. Cys residues not participating in the disulfide bridge formation are replaced by a Cys-acetamidomethyl residue.

For a twelfth library, bicyclic peptides of length 27 were generated. On positions 2-13 and 15-26 are 12-mer peptides derived from the sequence of CCL14. Cys residues are inserted on positions 1, 14 and 27 in order to create a discontinuous mimic by means of T3 CLIPS. Native Cys are replaced by a Cys-acetamidomethyl residue.

For a thirteenth library, bicyclic peptides of length 33 were generated. On positions 2-16 and 18-32 are 15-mer peptides derived from the sequence of CCL14. Cys residues are inserted on positions 1, 17 and 33 in order to create a discontinuous mimic by means of T3 CLIPS. Native Cys are replaced by a Cys-acetamidomethyl residue.

Antibodies were diluted in buffer and applied to the peptide library array. Each antibody tested was optimized for the array by testing different blocking conditions and sample concentration. Results were analyzed and binding events were noted with at least three times the median value. Epitope mapping was precluded in the case of antibodies showing high binding signals throughout the array.

Based on binding to the array, the following CCL14 epitopes were identified for antibodies of the present invention:

TABLE 3

| Clone | Epitope |
|---|---|
| 5H2/5K3 | SRGPYHPSECCFTYT (SEQ ID NO: 13) and |
| | YETNSQCSKPGIVFI (SEQ ID NO: 14) |
| 8H3/8K3 | YYETNSQCSKPGIVFI (SEQ ID NO: 15) |
| 9H3/9K2 | SDKWVQDYIKDMKE (SEQ ID NO: 16) |
| 14H1/14K1 | CCFTYTTYKIPRQR (SEQ ID NO: 17) and |
| | NSQCSKPGIVFIT (SEQ ID NO: 18) |
| 15H1/15K3 | TYKIPRQRIMDYYE (SEQ ID NO: 19) |
| and | |
| 24H1/24K1 | |

### Example 3: Antibody Pairing

Various antibodies of the invention were tested for their ability to form sandwich complexes in a standard sandwich enzyme immunoassay format. One member of the antibody pair (the “capture” antibody) which binds human CCL14 was immobilized in wells of a 96 well polystyrene plate. Human CCL14 standards or test samples (e.g. a body fluid sample) were pipetted into the appropriate wells and bound to the immobilized capture antibody. After washing away any unbound CCL14 or sample, a second CCL14 antibody (the “detection” antibody) was added to the wells, thereby forming sandwich complexes with the CCL14 (if present) and the capture antibody. After washing the wells to remove any unbound detection antibody, a goat anti-rabbit-horseradish peroxidase solution was added to the wells. The wells were washed to remove any unbound goat anti-rabbit-horseradish peroxidase and a substrate solution was added to the wells. Color develops in proportion to the amount of CCL14 present in the sample. The color development was stopped and the intensity of the color was measured at 540 nm. CCL14 antibodies tested included MAB3241 (a mouse monoclonal, R&D Systems), antibody B (mouse anti-CCL14 antibody) and antibody F (mouse anti-CCL14 antibody) as capture antibodies; and 5H2/5K3, 8H3/8K3, 9H3/9K2, 14H1/14K1, 15H1/15K3 and 24H1/24K1 as detection antibodies. The pairing results are shown in Table 4.

TABLE 4

| | MAB3241 | Antibody B | Antibody F |
|---|---|---|---|
| | Capture | Capture | Capture |
| 5H2/5K3 | Yes | Yes | Yes |
| Detection | | | |
| 8H3/8K3 | Yes | Yes | Yes |
| Detection | | | |
| 9H3/9K2 | No | No | No |
| Detection | | | |
| 14H1/14K1 | Yes | No | No |
| Detection | | | |
| 15H1/15K3 | No | No | No |
| Detection | | | |
| 24H1/24K1 | Yes | Yes | Yes |
| Detection | | | |

While the invention has been described and exemplified in sufficient detail for those skilled in this art to make and use it, various alternatives, modifications, and improvements should be apparent without departing from the spirit and scope of the invention. The examples provided herein are representative of preferred aspects, are exemplary, and are not intended as limitations on the scope of the invention. Modifications therein and other uses will occur to those skilled in the art. These modifications are encompassed within the spirit of the invention and are defined by the scope of the claims.

Unless specifically noted otherwise herein, the definitions of the terms used are standard definitions used in the art of pharmaceutical sciences. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

The use of “or” herein means “and/or” unless stated otherwise. Similarly, “comprise,” “comprises,” “comprising” “include,” “includes,” and “including” are interchangeable and not intended to be limiting.

It is to be further understood that where descriptions of various aspects use the term “comprising,” those skilled in the art would understand that in some specific instances, an aspect can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. All patents and publications mentioned in the specification are indicative of the levels of those of ordinary skill in the art to which the invention pertains prior to the filing date of the disclosure. Nothing herein is to be construed as an admission that the inventors are not entitled to antedate such disclosure by virtue of prior disclosure.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

What is claimed is:

1. An antibody which binds to an epitope on human CCL14, wherein the epitope comprises all or part of the sequence SRGPYHPSECCFTYT (SEQ ID NO: 13), YETNSQCSKPGIVFI (SEQ ID NO: 14), YYETNSQCSKPGIVFI (SEQ ID NO: 15), SDKWVQDYIKDMKE (SEQ ID NO: 16), CCFTYTTYKIPRQR (SEQ ID NO: 17), NSQCSKPGIVFIT (SEQ ID NO: 18), or TYKIPRQRIMDYYE (SEQ ID NO: 19).

2. An antibody which competes for binding to human C-C motif chemokine 14 (CCL14) protein with an antibody comprising:
(a) three complementarity determining regions (CDRs) of a heavy chain variable region set forth as SEQ ID NO: 1, and three CDRs of a light chain variable region set forth as SEQ ID NO: 2;
(b) three CDRs of a heavy chain variable region set forth as SEQ ID NO: 3, and three CDRs of a light chain variable region set forth as SEQ ID NO: 4;
(c) three CDRs of a heavy chain variable region set forth as SEQ ID NO: 5, and three CDRs of a light chain variable region set forth as SEQ ID NO: 6;
(d) three CDRs of a heavy chain variable region set forth as SEQ ID NO: 7, and three CDRs of a light chain variable region set forth as SEQ ID NO: 8;
(e) three CDRs of a heavy chain variable region set forth as SEQ ID NO: 9, and three CDRs of a light chain variable region set forth as SEQ ID NO: 10; or
(f) three CDRs of a heavy chain variable region set forth as SEQ ID NO: 11, and three CDRs of a light chain variable region set forth as SEQ ID NO: 12.

3. The antibody of claim 1 or 2, wherein the antibody comprises:
(a) three complementarity determining regions (CDRs) of a heavy chain variable region set forth as SEQ ID NO: 1, and three CDRs of a light chain variable region set forth as SEQ ID NO: 2;
(b) three CDRs of a heavy chain variable region set forth as SEQ ID NO: 3, and three CDRs of a light chain variable region set forth as SEQ ID NO: 4;
(c) three CDRs of a heavy chain variable region set forth as SEQ ID NO: 5, and three CDRs of a light chain variable region set forth as SEQ ID NO: 6;
(d) three CDRs of a heavy chain variable region set forth as SEQ ID NO: 7, and three CDRs of a light chain variable region set forth as SEQ ID NO: 8;
(e) three CDRs of a heavy chain variable region set forth as SEQ ID NO: 9, and three CDRs of a light chain variable region set forth as SEQ ID NO: 10; or
(f) three CDRs of a heavy chain variable region set forth as SEQ ID NO: 11, and three CDRs of a light chain variable region set forth as SEQ ID NO: 12.

4. The antibody of claim 1 or 2, wherein the antibody comprises:
(a) residues 27-38 of SEQ ID NO: 1 for CDR-H1, residues 56-65 of SEQ ID NO: 1 for CDR-H2, residues 105-117 of SEQ ID NO: 1 for CDR-H3, residues 27-38 of SEQ ID NO: 2 for CDR-L1, residues 56-65 of SEQ ID NO: 2 for CDR-L2 and residues 105-117 of SEQ ID NO: 2 for CDR-L3;
(b) residues 27-38 of SEQ ID NO: 3 for CDR-H1, residues 56-65 of SEQ ID NO: 3 for CDR-H2, residues 105-117 of SEQ ID NO: 3 for CDR-H3, residues 27-38 of SEQ ID NO: 4 for CDR-L1, residues 56-65 of SEQ ID NO: 4 for CDR-L2 and residues 105-117 of SEQ ID NO: 4 for CDR-L3;
(c) residues 27-38 of SEQ ID NO: 5 for CDR-H1, residues 56-65 of SEQ ID NO: 5 for CDR-H2, residues 105-117 of SEQ ID NO: 5 for CDR-H3, residues 27-38 of SEQ ID NO: 6 for CDR-L1, residues 56-65 of SEQ ID NO: 6 for CDR-L2 and residues 105-117 of SEQ ID NO: 6 for CDR-L3;
(d) residues 27-38 of SEQ ID NO: 7 for CDR-H1, residues 56-65 of SEQ ID NO: 7 for CDR-H2, residues 105-117 of SEQ ID NO: 7 for CDR-H3, residues 27-38 of SEQ ID NO: 8 for CDR-L1, residues 56-65 of SEQ ID NO: 8 for CDR-L2 and residues 105-117 of SEQ ID NO: 8 for CDR-L3;
(e) residues 27-38 of SEQ ID NO: 9 for CDR-H1, residues 56-65 of SEQ ID NO: 9 for CDR-H2, residues 105-117 of SEQ ID NO: 9 for CDR-H3, residues 27-38 of SEQ ID NO: 10 for CDR-L1, residues 56-65 of SEQ ID NO: 10 for CDR-L2 and residues 105-117 of SEQ ID NO: 10 for CDR-L3; or
(f) residues 27-38 of SEQ ID NO: 11 for CDR-H1, residues 56-65 of SEQ ID NO: 11 for CDR-H2, residues 105-117 of SEQ ID NO: 11 for CDR-H3, residues 27-38 of SEQ ID NO: 12 for CDR-L1, residues 56-65 of SEQ ID NO: 12 for CDR-L2 and residues 105-117 of SEQ ID NO: 12 for CDR-L3,
wherein the residues are numbered according to Lefranc.

5. The antibody of claim 1 or 2, wherein the antibody comprises:
(a) residues 31-35 of SEQ ID NO: 1 for CDR-H1, residues 50-65 of SEQ ID NO: 1 for CDR-H2 residues 95-102 of SEQ ID NO: 1 for CDR-H3, residues 24-34 of SEQ ID NO: 2 for CDR-L1, residues 50-56 of SEQ ID NO: 2 for CDR-L2, and residues 89-97 of SEQ ID NO: 2 for CDR-L3;
(b) residues 31-35 of SEQ ID NO: 3 for CDR-H1, residues 50-65 of SEQ ID NO: 3 for CDR-H2 residues 95-102 of SEQ ID NO: 3 for CDR-H3, residues 24-34 of SEQ ID NO: 4 for CDR-L1, residues 50-56 of SEQ ID NO: 4 for CDR-L2, and residues 89-97 of SEQ ID NO: 4 for CDR-L3;
(c) residues 31-35 of SEQ ID NO: 5 for CDR-H1, residues 50-65 of SEQ ID NO: 5 for CDR-H2 residues 95-102 of SEQ ID NO: 5 for CDR-H3, residues 24-34 of SEQ ID NO: 6 for CDR-L1, residues 50-56 of SEQ ID NO: 6 for CDR-L2, and residues 89-97 of SEQ ID NO: 6 for CDR-L3;
(d) residues 31-35 of SEQ ID NO: 7 for CDR-H1, residues 50-65 of SEQ ID NO: 7 for CDR-H2 residues 95-102 of SEQ ID NO: 7 for CDR-H3, residues 24-34 of SEQ ID NO: 8 for CDR-L1, residues 50-56 of SEQ ID NO: 8 for CDR-L2, and residues 89-97 of SEQ ID NO: 8 for CDR-L3;
(e) residues 31-35 of SEQ ID NO: 9 for CDR-H1, residues 50-65 of SEQ ID NO: 9 for CDR-H2 residues 95-102 of SEQ ID NO: 9 for CDR-H3, residues 24-34 of SEQ ID NO: 10 for CDR-L1, residues 50-56 of SEQ ID NO: 10 for CDR-L2, and residues 89-97 of SEQ ID NO: 10 for CDR-L3; or
(f) residues 31-35 of SEQ ID NO: 11 for CDR-H1, residues 50-65 of SEQ ID NO: 11 for CDR-H2 residues 95-102 of SEQ ID NO: 11 for CDR-H3, residues 24-34 of SEQ ID NO: 12 for CDR-L1, residues 50-56 of SEQ ID NO: 12 for CDR-L2, and residues 89-97 of SEQ ID NO: 12 for CDR-L3,
wherein the residues are numbered according to Kabat.

6. The antibody of claim 1 or 2, wherein the antibody comprises:
(a) residues 26-32 of SEQ ID NO: 1 for CDR-H1, residues 52-56 of SEQ ID NO: 1 for CDR-H2 residues 95-102 of SEQ ID NO: 1 for CDR-H3, residues 24-34 of SEQ ID NO: 2 for CDR-L1, residues 50-56 of SEQ ID NO: 2 for CDR-L2, and residues 89-97 of SEQ ID NO: 2 for CDR-L3;
(b) residues 26-32 of SEQ ID NO: 3 for CDR-H1, residues 52-56 of SEQ ID NO: 3 for CDR-H2 residues 95-102 of SEQ ID NO: 3 for CDR-H3, residues 24-34 of SEQ ID NO: 4 for CDR-L1, residues 50-56 of SEQ ID NO: 4 for CDR-L2, and residues 89-97 of SEQ ID NO: 4 for CDR-L3;
(c) residues 26-32 of SEQ ID NO: 5 for CDR-H1, residues 52-56 of SEQ ID NO: 5 for CDR-H2 residues 95-102 of SEQ ID NO: 5 for CDR-H3, residues 24-34 of SEQ ID NO: 6 for CDR-L1, residues 50-56 of SEQ ID NO: 6 for CDR-L2, and residues 89-97 of SEQ ID NO: 6 for CDR-L3;
(d) residues 26-32 of SEQ ID NO: 7 for CDR-H1, residues 52-56 of SEQ ID NO: 7 for CDR-H2 residues 95-102 of SEQ ID NO: 7 for CDR-H3, residues 24-34 of SEQ ID NO: 8 for CDR-L1, residues 50-56 of SEQ ID NO: 8 for CDR-L2, and residues 89-97 of SEQ ID NO: 8 for CDR-L3;
(e) residues 26-32 of SEQ ID NO: 9 for CDR-H1, residues 52-56 of SEQ ID NO: 9 for CDR-H2 residues 95-102 of SEQ ID NO: 9 for CDR-H3, residues 24-34 of SEQ ID NO: 10 for CDR-L1, residues 50-56 of SEQ ID NO: 10 for CDR-L2, and residues 89-97 of SEQ ID NO: 10 for CDR-L3; or
(f) residues 26-32 of SEQ ID NO: 11 for CDR-H1, residues 52-56 of SEQ ID NO: 11 for CDR-H2 residues 95-102 of SEQ ID NO: 11 for CDR-H3, residues 24-34 of SEQ ID NO: 12 for CDR-L1, residues 50-56 of SEQ ID NO: 12 for CDR-L2, and residues 89-97 of SEQ ID NO: 12 for CDR-L3,
wherein the residues are numbered according to Chothia.

7. The antibody of claim 1 or 2, wherein the antibody comprises:
(a) residues 30-35 of SEQ ID NO: 1 for CDR-H1, residues 47-58 of SEQ ID NO: 1 for CDR-H2 residues 93-101 of SEQ ID NO: 1 for CDR-H3, residues 30-36 of SEQ ID NO: 2 for CDR-L1, residues 46-55 of SEQ ID NO: 2 for CDR-L2, and residues 89-96 of SEQ ID NO: 2 for CDR-L3;
(b) residues 30-35 of SEQ ID NO: 3 for CDR-H1, residues 47-58 of SEQ ID NO: 3 for CDR-H2 residues 93-101 of SEQ ID NO: 3 for CDR-H3, residues 30-36 of SEQ ID NO: 4 for CDR-L1, residues 46-55 of SEQ ID NO: 4 for CDR-L2, and residues 89-96 of SEQ ID NO: 4 for CDR-L3;
(c) residues 30-35 of SEQ ID NO: 5 for CDR-H1, residues 47-58 of SEQ ID NO: 5 for CDR-H2 residues 93-101 of SEQ ID NO: 5 for CDR-H3, residues 30-36 of SEQ ID NO: 6 for CDR-L1, residues 46-55 of SEQ ID NO: 6 for CDR-L2, and residues 89-96 of SEQ ID NO: 6 for CDR-L3;
(d) residues 30-35 of SEQ ID NO: 7 for CDR-H1, residues 47-58 of SEQ ID NO: 7 for CDR-H2 residues 93-101 of SEQ ID NO: 7 for CDR-H3, residues 30-36 of SEQ ID NO: 8 for CDR-L1, residues 46-55 of SEQ ID NO: 8 for CDR-L2, and residues 89-96 of SEQ ID NO: 8 for CDR-L3;
(e) residues 30-35 of SEQ ID NO: 9 for CDR-H1, residues 47-58 of SEQ ID NO: 9 for CDR-H2 residues 93-101 of SEQ ID NO: 9 for CDR-H3, residues 30-36 of SEQ ID NO: 10 for CDR-L1, residues 46-55 of SEQ ID NO: 10 for CDR-L2, and residues 89-96 of SEQ ID NO: 10 for CDR-L3; or
(f) residues 30-35 of SEQ ID NO: 11 for CDR-H1, residues 47-58 of SEQ ID NO: 11 for CDR-H2 residues 93-101 of SEQ ID NO: 11 for CDR-H3, residues 30-36 of SEQ ID NO: 12 for CDR-L1, residues 46-55 of SEQ ID NO: 12 for CDR-L2, and residues 89-96 of SEQ ID NO: 12 for CDR-L3,
wherein the residues are numbered according to MacCallum.

8. The antibody of any one of claims 1-7, wherein the antibody comprises:
(a) a heavy chain variable region comprising an amino acid sequence set forth as SEQ ID NO: 1 and a light chain variable region comprising an amino acid sequence set forth as SEQ ID NO: 2;
(b) a heavy chain variable region comprising an amino acid sequence set forth as SEQ ID NO: 3 and a light chain variable region comprising an amino acid sequence set forth as SEQ ID NO: 4;
(c) a heavy chain variable region comprising an amino acid sequence set forth as SEQ ID NO: 5 and a light chain variable region comprising an amino acid sequence set forth as SEQ ID NO: 6;
(d) a heavy chain variable region comprising an amino acid sequence set forth as SEQ ID NO: 7 and a light chain variable region comprising an amino acid sequence set forth as SEQ ID NO: 8;
(e) a heavy chain variable region comprising an amino acid sequence set forth as SEQ ID NO: 9 and a light chain variable region comprising an amino acid sequence set forth as SEQ ID NO: 10; or
(f) a heavy chain variable region comprising an amino acid sequence set forth as SEQ ID NO: 11 and a light chain variable region comprising an amino acid sequence set forth as SEQ ID NO: 12.

9. The antibody of any one of claims 1-8, wherein the antibody is a rabbit antibody.

10. The antibody of any one of claims 1-9, wherein the antibody is a monoclonal antibody, a polyclonal antibody a humanized antibody; or an antigen binding fragment thereof.

11. The antibody of any one of claims 1-10, wherein the antibody is conjugated to a signal development element.

12. The antibody of any one of claims 1-10, wherein the antibody is immobilized on a solid support.

13. A nucleic acid encoding an amino acid heavy chain variable region and/or an amino acid light chain variable region of the antibody of any one of claims 1-10.

14. A vector or host cell comprising the nucleic acid of claim 13.

15. A kit comprising an antibody of any one of claims 1-12.

16. A kit comprising:
a first antibody of any one of claims 1-12, and a second antibody which specifically binds human CCL14, wherein the first antibody and the second antibody form a sandwich complex with human CCL14.

17. The kit of claim 16, wherein the second antibody is an antibody of any one of claims 1-12 that is a different antibody from the first antibody.

18. The kit of claim 16 or 17, further comprising a test device configured to generate a detectable signal related to the presence or amount of human CCL14 in a body fluid sample, wherein the first antibody or the second antibody is immobilized on a surface within the test device.

19. The kit of claim 18, wherein the test device is a disposable test device.

20. The kit of claim 18 or 19, wherein the test device is a lateral flow test device.

21. The kit of any one of claims 18-20, wherein the first antibody is immobilized to a surface within the test device, and the second antibody is conjugated to a detectable label.

22. The kit of any one of claims 18-20, wherein the first antibody is immobilized to a surface within the test device, and the second antibody is conjugated to a detectable label and is provided in a separate container from the test device.

23. The kit of any one of claims 18-22, wherein the kit further comprises a calibration curve to relate the detectable signal to a concentration of CCL14.

24. The kit of claim 23, wherein the calibration curve is provided on an electronic memory device.

25. The kit of any one of claims 18-22, wherein the kit further comprises reagents for generating a calibration curve.

26. The kit of any one of claims 16-25, wherein the kit is configured to perform an assay method which provides a signal related to the presence or amount of human CCL14 in a body fluid sample, and wherein the minimum detectable concentration of CCL14 in the assay method is 10 ng/mL or less.

27. The kit of any one of claims 16-26, wherein the second antibody is a monoclonal antibody, a polyclonal antibody, a humanized antibody, or an antigen binding fragment thereof.

28. The kit of any one of claims 16-27, wherein the second antibody is a rabbit antibody.

29. A kit comprising an antibody of any one of claims 1-12; and instructions for performing an immunoassay for CCL14.

30. The kit of claim 29, wherein the immunoassay is a competitive immunoassay.

31. A method for determining the presence or amount of human CCL14 in a body fluid sample, comprising:
performing an immunoassay on the body fluid sample with a first antibody and a second antibody which together form a sandwich complex with human CCL14, wherein the immunoassay provides a detectable signal that is related to a presence or an amount of human CCL14 in the body fluid sample bound in the sandwich complex; and
relating the detectable signal to the presence or amount of human CCL14 in the body fluid sample, wherein the first antibody, and optionally the second antibody, is an antibody according to any one of claims 1-12.

32. The method of claim 31, wherein the minimum detectable concentration of CCL14 in the immunoassay is 10 ng/mL or less.

33. The method of claim 31 or 32, wherein the immunoassay is performed in a lateral flow format.

34. The method of any one of claims 31-33, wherein the immunoassay is performed by applying the body fluid sample to a test device, and the detectable signal is obtained by inserting the test device into an analytical instrument, wherein the sandwich complex comprising the first and second antibodies is immobilized for detection in a predetermined zone of the test device, and wherein the analytical instrument detects the immobilized sandwich complex to provide the detectable signal.

35. The method of claim 34, wherein the test device is a disposable test device.

36. The method of any one of claims 31-35, wherein the first antibody is conjugated to a signal development element.

37. The method of claim 36, wherein the first antibody forms a reaction mixture with the body fluid sample, and the body fluid sample is applied to the test device by applying the reaction mixture to the test device.

38. The method of claim 36 or 37, wherein the second antibody is immobilized at the predetermined zone of a solid support.

39. The method of any one of claims 31-38, wherein each of the first and second antibodies is a rabbit or a mouse antibody or antigen binding fragment thereof.

40. The method of claim 39, wherein at least one of the first and second antibodies is a rabbit antibody or antibody fragment.

41. The method of any one of claims 31-40, wherein one or both of the first or second antibodies is a monoclonal antibody, a polyclonal antibody, a humanized antibody or an antigen binding fragment thereof.

42. A method for determining the presence or amount of human CCL14 in a body fluid sample, comprising:
performing a competitive immunoassay on the body fluid sample with an antibody which binds human CCL14, wherein the competitive immunoassay provides a detectable signal that is related to the presence or amount of human CCL14 in the body fluid sample; and
relating the detectable signal to the presence or amount of human CCL14 in the body fluid sample, wherein the antibody is an antibody of any one of claims 1-12.
